(19) United States
(12) Patent Application Publication
(10) Pub. No.: US 2025/0304673 A1
(43) Pub. Date: Oct. 2, 2025

(54) ANTI-GDF15 ANTIBODY USED IN A COMBINATION TREATMENT OF SPECIFIC PATIENT GROUPS AND A DOSAGE REGIMEN FOR THE TREATMENT OF CANCER

(71) Applicant: CatalYm GmbH, Planegg-Martinsried (DE)

(72) Inventors: Kathrin Klar, Munich (DE); Eugen Leo, Munich (DE)

(21) Appl. No.: 19/074,614

(22) Filed: Mar. 10, 2025

Related U.S. Application Data
(63) Continuation of application No. PCT/EP2023/074738, filed on Sep. 8, 2023.

(30) Foreign Application Priority Data
Sep. 8, 2022 (EP) 22194590

(51) Int. Cl. C07K 16/22; A61K 39/00; A61P 35/00; C07K 16/28; G01N 33/574; G01N 33/68

(52) U.S. Cl. CPC C07K 16/22; A61P 35/00; C07K 16/2818; C07K 16/2827; C07K 16/2878; G01N 33/57407; G01N 33/57492; G01N 33/6893; A61K 2039/507; A61K 2039/54; A61K 2039/545; C07K 2317/14; C07K 2317/565; G01N 2333/70532; G01N 2474/20

(57) ABSTRACT

The present invention relates to the treatment of specific patient groups using an anti-GDF15 antibody in combination with a checkpoint inhibitor. Furthermore, the invention also provides a dosage regimen for the treatment of cancers in a human patient using an anti-GDF15 antibody.

### CROSS-REFERENCE TO RELATED APPLICATIONS

This application is a continuation of International Patent Application No. PCT/EP2023/074738, filed Sep. 8, 2023, which claims priority from European Application No. 22194590, filed Sep. 8, 2022. The contents of these applications are incorporated herein by reference in their entireties.

### SEQUENCE LISTING

The content of the electronically submitted Sequence Listing XML (Name: 216648_SL.xml; Size: 34,652 bytes; Created on Mar. 7, 2025) is incorporated by reference herein in its entirety.

### FIELD OF INVENTION

The present invention relates to the treatment of specific patient groups using an anti-GDF15 antibody in combination with a checkpoint inhibitor. Furthermore, the invention also provides a dosage regimen for the treatment of cancers in human subjects using an anti-GDF15 antibody.

### BACKGROUND

GDF-15 is a divergent member of the TGF-beta superfamily for which functions in appetite regulation, metabolism, inflammation, cell and tissue survival, and immune tolerance have been described. GDF-15 is a homodimer, that is generated as a pro-protein, which is cleaved to a 25 kDa (2×112 aa) dimeric mature GDF-15 and 2×18 kDa (2×167 aa) pro-peptides that reside in the tissue (Tsai 2018) and are released into the bloodstream.

To date two main categories of activity of GDF-15 have been described. The first category relates to a metabolic effect, i.e. GDF-15 mediates cachexia via changing food-intake behavior, inducing anorexia. (Johnen 2007) This effect is mediated by a brain stem specific receptor named GFRAL which was described in late 2017 (Emmerson 2017). In contrast, the second category relates to an immunomodulatory effect, i.e. GDF-15 was shown to be a mediator of immune tolerance in pregnancy (Tong et al. 2004), tissue injury (Chung et al. 2017) and inflammation (Abulizi 2016), auto-immune diseases and tumor evasion. GDF-15 inhibits leukocyte integrin activation and thereby prevents their infiltration (Kempf 2011).

In recent years increasing evidence has emerged that GDF-15 seems to play a critical immuno-regulatory role in physiologic and pathophysiologic situations and specifically in cancer. For cancer cells it would naturally be highly attractive to utilize and “hijack” such an immune-cell repellant mechanism, blocking immune-cell entry into the tumor microenvironment, and consequently preventing the immune system from removing cancer cells. In line with this, in recent years a wealth of publications has emerged indicating that high GDF-15 serum levels in various cancer types correlate with shorter overall survival and that GDF-15 is an independent factor for patient survival within various tumor types (Wischhusen et al, 2020).

Elevated GDF-15 levels are frequently reported in cancer patients. In a microarray-based study comparing 150 carcinomas from 10 anatomic sites of origin with 46 normal tissues GDF-15 showed the highest level of tumor-associated (over)expression (Welsh 2003) and several studies correlate GDF-15 serum levels and lower response/worse prognosis in cancer.

In addition, two proprietary analyses with two different academic melanoma study groups indicate that GDF-15 levels prior to treatment also seem to correlate with response to PD-1 antagonists.

As indicated, cancer tissues, normal organ tissues in distress and placenta are known to overexpress GDF-15, presumably to prevent an excessive immune cell infiltration to the respective tissue. Hence, the inventors considered that GDF-15 produced by above tissues does substantially reduce vascular T cell adhesion and endothelial transmigration, preventing T cell entry into the respective tissue or its immediate proximity.

Whilst an anti-GDF-15 antibody generally shows a benign and well acceptable safety profile in animal models, this mode-of-action naturally carries various potential risks when aiming at providing a suitable dosage regimen for the treatment of humans.

Rational combination partners for an anti-GDF-15 antibody will be T-cell activating compounds, such as anti-PD-1/PD-L1 checkpoint inhibitors, or cellular T cell therapies such as CAR-T or TCR-T therapies. The efficacy of such compounds and treatments may be substantially enhanced by increasing intratumoral presence of antitumoral T-cells. Yet, potentially also their toxicities may be potentiated when combining them with certain dosage regimens of an anti-GDF-15 neutralizing antibody.

WO 2022/101263 describes an anti-GDF-15 antibody and a dosage regimen for the treatment of cancer.

A second potential area of concern is the physiologic role of GDF-15 in organ protection for organs in distress. If GDF-15 is suppressed using an anti-GDF-15 antibody and organ distress occurs (e.g. myocardial infarction, infection, other significant organ damage) excessive organ infiltration by immune cells and unwelcome tissue impairment/destruction might occur.

A third potential area of concern are rare findings made in individual mouse knock-out models for GDF-15 (Wischhusen 2020).

A further challenge in the field of cancer therapy is the identification of subtypes of cancers which are particularly responsive to the respective cancer therapy. While the average therapeutic effect of a particular cancer therapy on a cancer may only be moderate, particular subtypes of the respective cancer and the respective subgroup of cancer patients may be particularly responsive to the therapy and may therefore face a more favourable clinical situation than the other patients. In fact, growing evidence suggests that for many types of cancers, there exist distinct cancer subtypes (especially molecular and/or histologic subtypes) with distinct prognostic and therapeutic implications.

These considerations apply particularly to targeted cancer therapies, i.e. therapies relying on the inhibition of a particular target protein. Since the treatment with anti-GDF-15 antibodies is a targeted therapy, there is a need for the identification of types and subtypes of cancers which are particularly responsive to the therapy with anti-GDF-15 antibodies.

Similarly, there also remains a challenge in the field of cancer therapy to direct the treatment to (a) patient group(s) which is particularly responsive to the therapy. Generally, the characterisation of a patient group may be based on various factors such as the patient's treatment history or the presence of biological markers within the cancer tissue. In this respect, the immune checkpoint molecules or PD-L1 have previously been considered as a potential marker for cancer treatment using anti-PD1 or anti-PD-L1 antibodies. However, a particular need arises for the identification of suitable biomarkers for combination treatments with anti-GDF-15 antibodies and immune checkpoint inhibitors such as anti-PD1 or anti-PD-L1 antibodies. For example, such combination treatments can be used successfully even in patients who are refractory to a monotherapy with anti-PD1 or anti-PD-L1 antibodies or to combination therapies with anti-PD1 or anti-PD-L1 antibodies and other anticancer agents. Thus, such combination therapies are clinically different from a monotherapy with anti-PD1 or anti-PD-L1 antibodies or from combination therapies with anti-PD1 or anti-PD-L1 antibodies and other anticancer agents, and they are expected to be associated with different biomarkers. Consequently, there remains the need in the art to allow the identification/enrichment of specific patient groups which are particularly responsive to cancer therapy, and to allow the treatment of these patient groups.

### DESCRIPTION OF INVENTION

In order to meet the above needs, the present invention provides a treatment for specific patient subgroups which are particularly responsive to the therapy using a checkpoint inhibitor in combination with an anti-GDF-15 antibody.

In a further aspect, the invention also provides a safe and effective dosage regimen of anti-GDF-15 antibodies in the therapeutic treatment of such patient subgroups.

According to the invention, these patient subgroups will particularly benefit from the treatment with the anti-GDF-15 antibodies.

Originally, PD-L1 was described as a marker to enrich for patients to respond to aPD-1/-L1 monotherapy. The inventors have, inter alia, now found that patients who previously relapsed or were refractory to anti-PD-1/L-1 therapy and had tumors expressing PD-L1 at baseline (i.e. at the time when the treatment with anti-GDF-15 antibodies was started) and/or having signs for an inflamed tumor at baseline (as reflected, for instance, by a high Tumor Inflammation Score (TIS) and certain minimum CD3 or CD8-cell infiltration counts) are more likely to have a particularly favourable clinical outcome (e.g., a treatment response) when treated with anti-GDF-15 antibodies in a combination therapy with anti-PD1/anti-PD-L1 antibodies such as nivolumab as compared to patients with tumors which do not have these properties.

It was previously reported that baseline samples obtained from anti-PD1/anti-PD-L1 (monotherapy) responder patients showed higher numbers of CD8-, PD-1- and PD-L1-expressing cells in the tumours (Tumeh et al., Nature. 2014 Nov. 27; 515(7528):568-71). Additionally, it was also reported that the TIS correlates with the expression of PD-L1, which is also known as CD274 (Danaher et al., 2018).

However, the patients, who were subject to the studies of the present inventors, were patients who had progressive disease when treated with anti-PD1 or anti-PD-L1 antibodies. Under these circumstances where the therapy using anti-PD1 or anti-PD-L1 antibodies had already been proven to fail in the monotherapeutic setting, it was highly unexpected that patients with cancers having high TIS, certain minimum CD3 or CD8-cell infiltration counts and PD-L1 expression would particularly benefit from a combination therapy of an anti-GDF-15 antibody with checkpoint blockers, e.g., anti-PD1/anti-PD-L1 antibodies such as nivolumab. Rather, it was expected that the clinical outcome of an immunotherapy with an anti-GDF-15 antibody in combination with checkpoint inhibitors would be governed by entirely different biomarkers.

Furthermore, although inhibition of GDF-15 by an anti-GDF-15 antibody helps to increase the percentage of CD3+ and CD8+ T-cells in the cancer, as has already been described previously, for instance, in WO 2017/055613, it was unexpectedly found by the present inventors that a pre-existence of CD3+ and CD8+ cells and PD-L1 in the cancers, as well as a high TIS score, is nonetheless beneficial for the clinical outcome of the treatment. In some aspects of the invention, for example, the baseline infiltration documents a residual antitumoral immune response which then can be enhanced by anti-GDF-15 and kept active by combination therapy of anti-GDF-15 with checkpoint inhibitors such as anti-PD-1/-L1 to ultimately result in tumor shrinkage.

The selection of patient groups with a more favorable clinical outcome according to the present invention is expected to be a helpful and effective selection criterion in clinical practice, because only a subset of the patients has cancers with such properties. For example, while PD-L1 can be expressed in many different cancer types, only a sub-group of cancer patients of each cancer type will express PD-L1 (see Yarchoan et al., JCI Insight. 2019 Mar. 21; 4(6):e126908 and Chen et al., Exp Hematol Oncol. 2020 Aug. 3; 9:17. doi: 10.1186/s40164-020-00173-3. eCollection 2020.).

Accordingly, the present invention allows, inter alia, to select, and to selectively treat, patients who will be in a particularly favorable clinical situation when treated with a combination therapy of an anti-GDF-15 antibody with checkpoint blockers, e.g., anti-PD1/anti-PD-L1 antibodies such as nivolumab.

According to the present invention, analysis of clinical data revealed inter alia PD-L1 TPS as positive enrichment factor for clinical response. Moreover, clinical data also revealed that patient selection by PD-L1 TPS alone or a combination of PD-L1 TPS with any of CD8 cell density, CD3 cell density or TIS enriched for clinical responder.

Accordingly, the present invention provides the following preferred embodiments:

Item 1 A pharmaceutical composition comprising a combination of an anti-GDF-15 antibody and at least one checkpoint inhibitor for use in a method of treating cancer in a human patient, wherein the human patient has a PD-L1 positive cancer and/or has a cancer comprising an inflamed tumor.

Item 2 The pharmaceutical composition for use according to item 1, wherein the cancer is PD-L1 positive as determinable by an immunohistochemistry (IHC) assay.

Item 3 The pharmaceutical composition for use according to item 2, wherein the IHC assay uses an anti-PD-L1 antibody as primary antibody and determines the percentage of PD-L1 positive cancer cells.

Item 4 The pharmaceutical composition for use according to any one of items 2 to 3, wherein the PD-L1 positive cancer has a percentage of cancer cells showing staining for the presence of PD-L1 on the cell surface of at least 1 in the immunohistochemistry (IHC) assay.

Item 5 The pharmaceutical composition for use according to item 2, wherein the IHC assay uses an anti-PD-L1 antibody as primary antibody and determines the summed-up percentage of PD-L1 positive cancer cells and PD-L1 positive cancer-infiltrating cells.

Item 6 The pharmaceutical composition for use according to any one of items 1 to 5, wherein the inflamed tumor is a T-cell inflamed tumor.

Item 7 The pharmaceutical composition for use according to any one of items 1 to 6, wherein the inflamed tumor is a tumor which contains CD3+ cells.

Item 8 The pharmaceutical composition for use according to item 7, wherein the inflamed tumor contains CD3+ cells as determinable by an immunohistochemistry (IHC) assay, and wherein the IHC assay uses an anti-CD3 antibody as primary antibody.

Item 9 The pharmaceutical composition for use according to item 8, wherein the inflamed tumor is a tumor having an average CD3+ cell density of >300 cells/mm² in immunohistochemical tissue sections having a thickness of 4 μm, as assessed by the immunohistochemistry (IHC) assay.

Item 10 The pharmaceutical composition for use according to any one of items 1 to 9, wherein the inflamed tumor is a tumor which contains CD8+ cells.

Item 11 The pharmaceutical composition for use according to item 10, wherein the inflamed tumor contains CD8+ cells as determinable by an immunohistochemistry (IHC) assay, and wherein the IHC assay uses an anti-CD8 antibody as primary antibody.

Item 12 The pharmaceutical composition for use according to item 11, wherein the inflamed tumor is a tumor having an average CD8+ cell density of >300 cells/mm² in immunohistochemical tissue sections having a thickness of 4 μm, as assessed by the immunohistochemistry (IHC) assay.

Item 13 The pharmaceutical composition for use according to any one of items 6 to 12, wherein the T-cell inflamed tumor is inflamed as indicated by its tumor inflammation signature (TIS).

Item 14 The pharmaceutical composition for use according to item 13, wherein the TIS is based on the expression of at least one, preferably all, of the marker genes selected from the group consisting of PSMB10, HLA-DQA1, HLA-DRB1, CMKLR1, HLA-E, NKG7, CD8A, CCL5, CXCL9, CD27, CXCR6, IDO1, STAT1, TIGIT, LAG3, CD274, PDCD1LG2 and CD276.

Item 15 The pharmaceutical composition for use according to item 14, wherein the TIS is a linear combination of all of said marker genes, calculated as

$$T I S = {\sum }_{i = 1}^{18} {x}_{i} {w}_{i} ,$$

where xᵢ is the i^{th} gene's log 2-transformed, normalized expression level and wᵢ is a predefined weight above zero.

Item 16 The pharmaceutical composition for use according to item 15, wherein the TIS is greater than 7.5.

Item 17 The pharmaceutical composition for use according to any one of items 1 to 16, wherein the human patient is an anti-PD-1 and/or anti-PD-L1 relapsed and/or refractory patient.

Item 18 The pharmaceutical composition for use according to any one of items 1 to 17, wherein the checkpoint inhibitor is selected from one or more of the group consisting of anti-PD-1 antibody or a PD-1-binding fragment thereof, anti-PD-L1 antibody or a PD-L1-binding fragment thereof, anti-CD40 antibody or a CD40-binding fragment thereof, anti-LAG-3 antibody or a LAG-3-binding fragment thereof, anti-TIM-3 antibody or a TIM-3-binding fragment thereof, anti-TIGIT antibody or a TIGIT-binding fragment thereof and anti-CTLA4 antibody or a CTLA4-binding fragment thereof.

Item 19 The pharmaceutical composition for use according to item 18, wherein the checkpoint inhibitor is selected from the group consisting of anti-PD-1 antibody or a PD-1-binding fragment thereof and anti-PD-L1 antibody or a PD-L1-binding fragment thereof.

Item 20 The pharmaceutical composition for use according to any one of items 1 to 19, wherein the cancer is a solid cancer.

Item 21 The pharmaceutical composition for use according to any one of items 1 to 20, wherein the cancer is selected from the group consisting of the group consisting of colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP) and breast carcinoma.

Item 22 The pharmaceutical composition for use according to any one of items 1 to 21, wherein the anti-GDF-15 antibody comprises a heavy chain variable domain comprising a CDR1 region represented by an amino acid sequences shown in SEQ ID NO: 1, a CDR2 region represented by an amino acid sequences shown in SEQ ID NO: 2 and a CDR3 region represented by an amino acid sequences shown in SEQ ID NO: 3 and a light chain variable domain comprising a CDR1 region represented by an amino acid sequences shown in SEQ ID NO: 4, a CDR2 region represented by an amino acid sequence ser-ala-ser and a CDR3 region represented by an amino acid sequences shown in SEQ ID NO: 5.

Item 23 The pharmaceutical composition for use according to any one of items 1 to 22, wherein the anti-GDF-15 antibody has a heavy chain variable domain comprising the amino acid sequence represented by SEQ ID NO: 6 or an amino acid sequence having at least 90% identity, preferably at least 95% identity and more preferably at least 98% identity to the amino acid sequence shown in SEQ ID NO: 6 and a light chain variable domain comprising the amino acid sequence represented by SEQ ID NO: 7 or an amino acid sequence having at least 90% identity, preferably at least 95% identity and more preferably at least 98% identity to the amino acid sequence shown in SEQ ID NO: 7.

Item 24 The pharmaceutical composition for use according to any one of items 1 to 23, wherein the anti-GDF-15 antibody has a heavy chain comprising the amino acid sequence represented by SEQ ID NO: 8 or an amino acid sequence having at least 90% identity, preferably at least 95% identity and more preferably at least 98% identity to the amino acid sequence shown in SEQ ID NO: 8 and a light chain comprising the amino acid sequence represented by SEQ ID NO: 9 or an amino acid sequence having at least 90% identity, preferably at least 95% identity and more preferably at least 98% identity to the amino acid sequence shown in SEQ ID NO: 9.

Item 25 The pharmaceutical composition for use according to any one of items 1 to 24, wherein the anti-GDF-15 antibody has a heavy chain comprising the amino acid sequence represented by SEQ ID NO: 8 and a light chain comprising the amino acid sequence represented by SEQ ID NO: 9.

Item 26 The pharmaceutical composition for use according to any one of items 1 to 25, wherein the anti-GDF-15 antibody is to be administered to the human patient, preferably
i) at a dose of between 3 and 20 mg/kg, preferably at a dose of 10 mg/kg, and at a dosage regimen of at least one administration cycle, wherein the cycle is a period of two weeks and wherein said dose is to be administered at least once in each of the at least one cycle,
ii) at a dose of between 3 and 20 mg/kg, preferably at a dose of 10 mg/kg, and at a dosage regimen of at least one administration cycle, wherein the cycle is a period of three weeks and wherein said dose is to be administered at least once in each of the at least one cycle, or
iii) at a dose of between 3 and 20 mg/kg, preferably at a dose of 20 mg/kg, and at a dosage regimen of at least one administration cycle, wherein the cycle is a period of four weeks and wherein said dose is to be administered at least once in each of the at least one cycle.

and wherein the cancer is selected from the group consisting of colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP) and breast carcinoma.

Item 27 The pharmaceutical composition for use according to any one of items 1 to 26, wherein the cancer is colorectal cancer.

Item 28 The pharmaceutical composition for use according to any one of items 1 to 26, wherein the cancer is bladder cancer.

Item 29 The pharmaceutical composition for use according to any one of items 1 to 26, wherein the cancer is cancer of unknown primary (CUP).

Item 30 The pharmaceutical composition for use according to any one of items 1 to 26, wherein the cancer is mesothelioma.

Item 31 The pharmaceutical composition for use according to any one of items 1 to 26, wherein the cancer is cutaneous squamous cell cancer.

Item 32 The pharmaceutical composition for use according to any one of items 1 to 26, wherein the cancer is non-small cell lung cancer.

Item 33 The pharmaceutical composition for use according to any one of items 1 to 26, wherein the cancer is hepatocellular cancer.

Item 34 The pharmaceutical composition for use according to any one of items 1 to 26, wherein the cancer is melanoma.

Item 35 The pharmaceutical composition for use according to any one of the preceding items, wherein the use is also a use for the treatment of cancer-cachexia.

Item 36 The pharmaceutical composition for use according to any one of items 1 to 35, wherein the anti-GDF-15 antibody is to be administered at a dose of between 3 and 20 mg/kg, preferably 20 mg/kg, and at a dosage regimen of at least one administration cycle, wherein the cycle is a period of four weeks and wherein said dose is to be administered at least once in each of the at least one cycle.

Item 37 The pharmaceutical composition for use according to any one of items 1 to 36, wherein the anti-GDF-15 antibody is to be administered at a dose of between 3 and 20 mg/kg, preferably 10 mg/kg, and at a dosage regimen of at least one administration cycle, wherein the cycle is a period of three weeks and wherein said dose is to be administered at least once in each of the at least one cycle.

Item 38 The pharmaceutical composition for use according to any one of items 1 to 37, wherein the anti-GDF-15 antibody is to be administered at a dose of between 3 and 20 mg/kg, preferably 10 mg/kg, and at a dosage regimen of at least one administration cycle, wherein the cycle is a period of two weeks and wherein said dose is to be administered at least once in each of the at least one cycle.

Item 39 The pharmaceutical composition for use according to any one of items 1 to 38, wherein said anti-GDF-15 antibody is obtainable by expression in CHO cells.

Item 40 The pharmaceutical composition for use according to any one of items 1 to 39, wherein said checkpoint inhibitor is to be administered in the same dosage regimen as the anti-GDF-15 antibody.

Item 41 The pharmaceutical composition for use according to any one of items 1 to 40, wherein said checkpoint inhibitor is to be administered prior to the administration of anti-GDF-15 antibody, preferably within 120 min prior to the administration of anti-GDF-15 antibody, more preferably within 30 minutes prior to the administration of anti-GDF-15 antibody.

Item 42 The pharmaceutical composition for use according to any one of items 1 to 41, wherein said dose of the anti-GDF-15 antibody is to be administered intravenously.

Item 43 A method for predicting the clinical outcome of a human cancer patient to a treatment comprising a combination of an anti-GDF-15 antibody and at least one checkpoint inhibitor, wherein the method comprises the steps of
(i) analyzing whether the cancer is a PD-L1 positive cancer and/or is a cancer comprising an inflamed tumor, and
(ii) predicting the clinical outcome of the patient to the treatment based on the analysis.

Item 44 Method of treating a human patient diagnosed with cancer comprising the steps of
(a) analyzing whether the cancer is a PD-L1 positive cancer and/or is a cancer comprising an inflamed tumor,
(b) predicting the clinical outcome of the patient for the treatment based on the analysis, and
(c) treating the human patient comprising a combination of an anti-GDF-15 antibody and at least one checkpoint inhibitor.

Item 45 The method according to item 43 or 44, wherein the clinical outcome comprises response, complete response, partial response, stable disease, progressive disease and/or survival of a human cancer patient.

Item 46 The method according to any one of items 43 to 45, wherein the patient is predicted to show improved clinical outcome if the cancer is PD-L1 positive.

Item 47 The method according to item 46, wherein the PD-L1 positive cancer is as defined in items 2-5.

Item 48 The method according to any one of items 43 to 47, wherein the patient is predicted to show improved clinical outcome if the cancer comprises an inflamed tumor.

Item 49 The method according to item 48, wherein the inflamed tumor is as defined in items 6-16.

Item 50 The method according to any one of items 43 to 49, wherein the checkpoint inhibitor is as defined in item 18 or 19.

Item 51 The method according to any one of items 43 to 50, wherein the cancer is selected from a cancer as defined in item 20 or 21.

Item 52 The method according to any one of items 43 to 51, wherein the human patient is an anti-PD-1 and/or anti-PD-L1 relapsed and/or refractory patient.

Item 53 An anti-GDF-15 antibody for use in a method of treating cancer in a human patient, wherein said anti-GDF-15 antibody is to be administered in combination with at least one checkpoint inhibitor, and wherein the human patient has a PD-L1 positive cancer and/or has a cancer comprising an inflamed tumor.

Item 54 The anti-GDF-15 antibody for use according to item 53, wherein the patient has a cancer as defined in any one of items 2-16, 19-21 and 26-34.

Item 55 The anti-GDF-15 antibody for use according to item 53 or 54, wherein the patient is as defined in item 17.

Item 56 The anti-GDF-15 antibody for use according to any one of items 53-55, wherein the checkpoint inhibitor is as defined in any one of items 18-19.

Item 57 The anti-GDF-15 antibody for use according to any one of items 53-56, wherein the anti-GDF-15 antibody is as defined in any one of items 22-25 and 39.

Item 58 The anti-GDF-15 antibody for use according to any one of items 53-57, wherein the anti-GDF-15 antibody is to be administered as defined in any one of items 26, 36-38 and 42.

Item 59 The anti-GDF-15 antibody for use according to any one of items 53-58, wherein the checkpoint inhibitor is to be administered as defined in any one of items 40-41.

Item 60 The anti-GDF-15 antibody for use according to any one of items 53-59, wherein the use is also a use for the treatment of cancer-cachexia.

Item 61 The pharmaceutical composition for use according to any one of items 1 to 42 or the anti-GDF-15 antibody for use according to any one of items 53 to 60, wherein the PD-L1 positive cancer has a percentage of cancer cells showing staining for the presence of PD-L1 on the cell surface of at least 1, preferably of at least 2, more preferably of at least 5 in the immunohistochemistry (IHC) assay.

Item 62 The pharmaceutical composition or the anti-GDF-15 antibody for use according to item 61, wherein the cancer is bladder cancer.

It is understood that in accordance with the present invention, references to “at least one checkpoint inhibitor” mean that the indicated checkpoint inhibitor is required, and that additional checkpoint inhibitors can be included or not, i.e., they are optionally included. For example, it is understood that in cases where the present invention refers to at least one checkpoint inhibitor wherein the checkpoint inhibitor is selected from the group consisting of anti-PD-1 antibody or a PD-1-binding fragment thereof and anti-PD-L1 antibody or a PD-L1-binding fragment thereof, this means that a checkpoint inhibitor selected from the group consisting of anti-PD-1 antibody or a PD-1-binding fragment thereof and anti-PD-L1 antibody or a PD-L1-binding fragment thereof is required, and that additional checkpoint inhibitors (e.g., anti-CD40 antibody or a CD40-binding fragment thereof, anti-LAG-3 antibody or a LAG-3-binding fragment thereof, anti-TIM-3 antibody or a TIM-3-binding fragment thereof, anti-TIGIT antibody or a TIGIT-binding fragment thereof and anti-CTLA4 antibody or a CTLA4-binding fragment thereof) can be included or not, i.e., they are optionally included.

### BRIEF DESCRIPTION OF THE DRAWINGS

FIG. 1A-FIG. 1C: Treatment of GDF-15-producing tumors with anti-mGDF-15 antibody substantially improves response to anti-PD1 antibodies.

Anti-mGDF-15 antibody alone had moderate effect, whereas combination with anti-PD-1 was able to substantially improve the therapeutic effect of anti-PD-1 treatment.

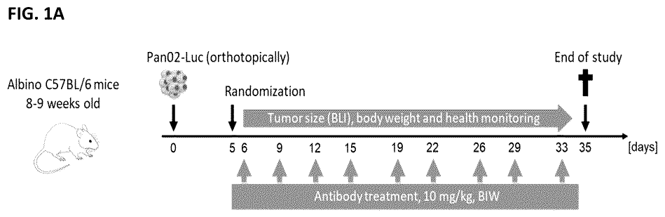

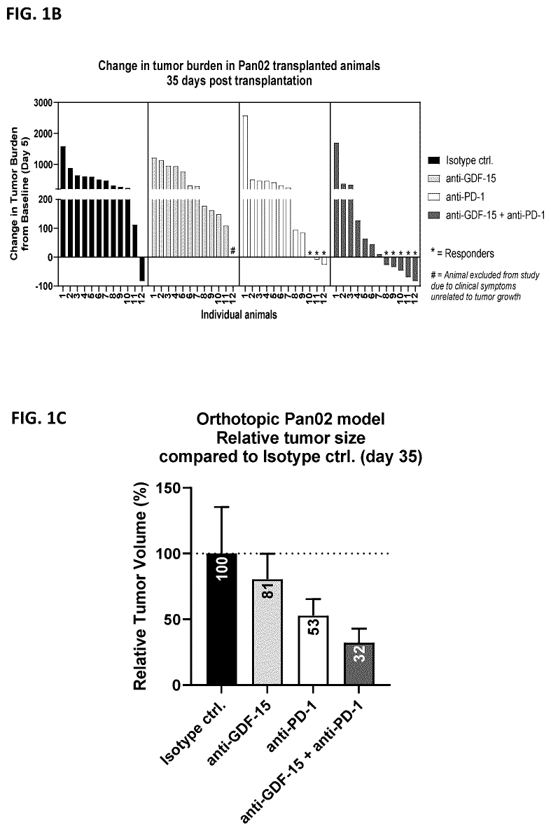

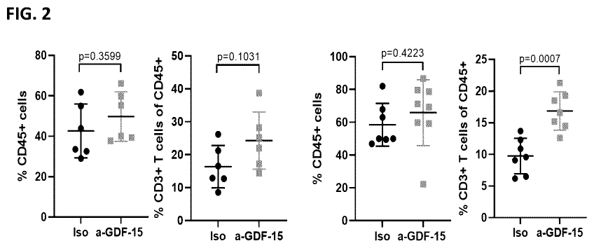

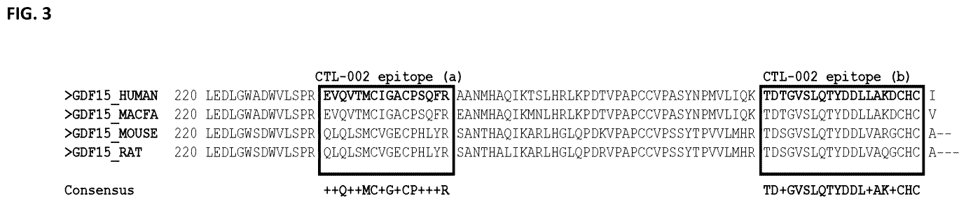

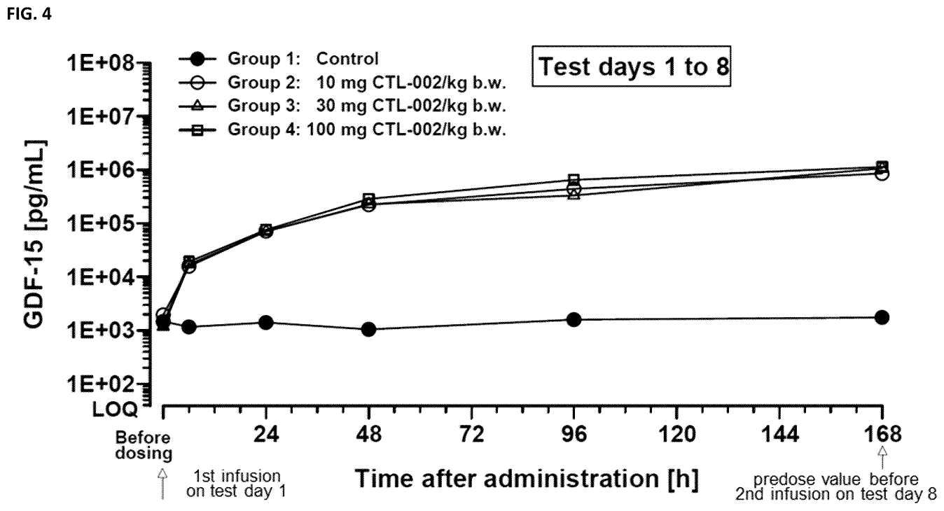

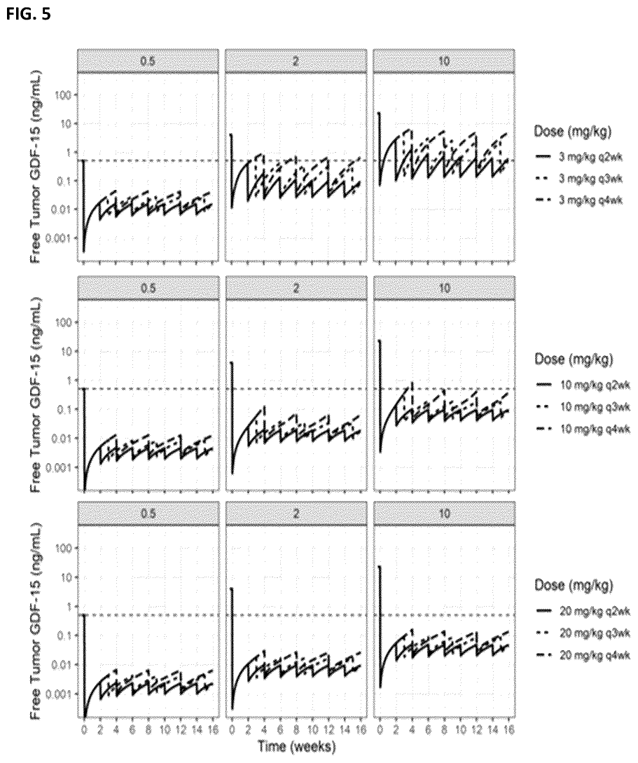

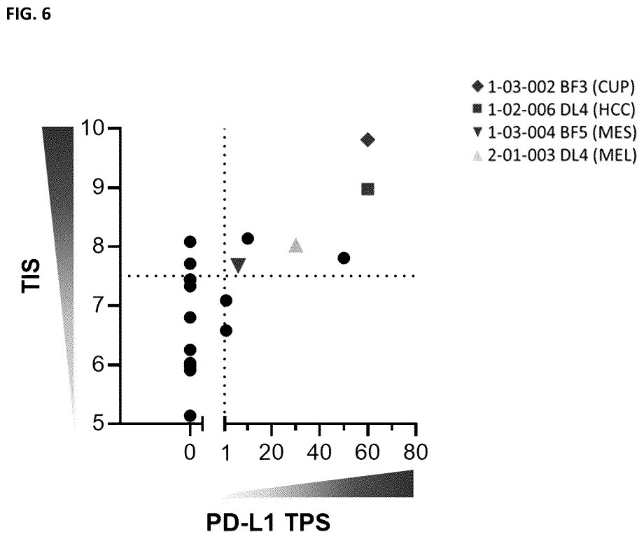

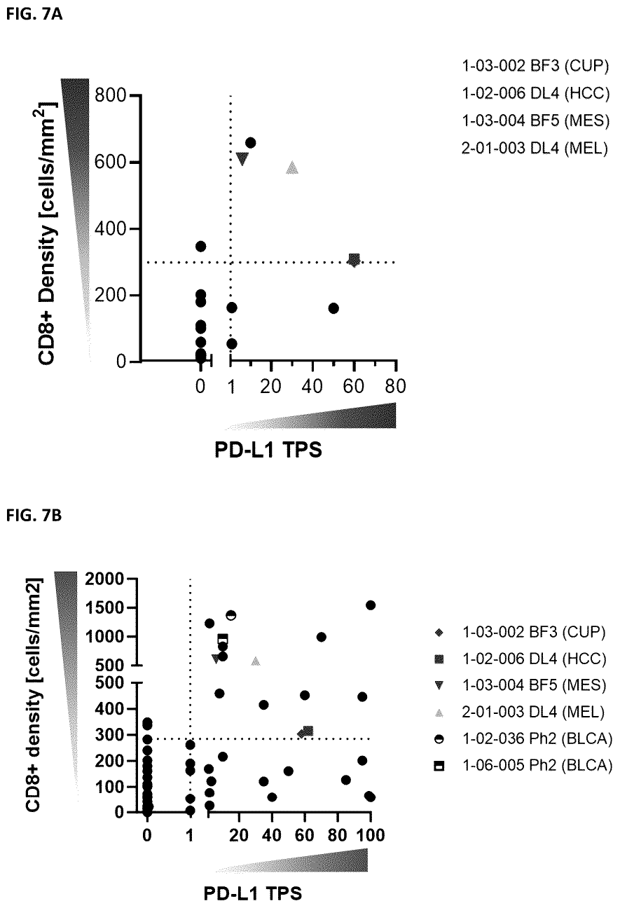

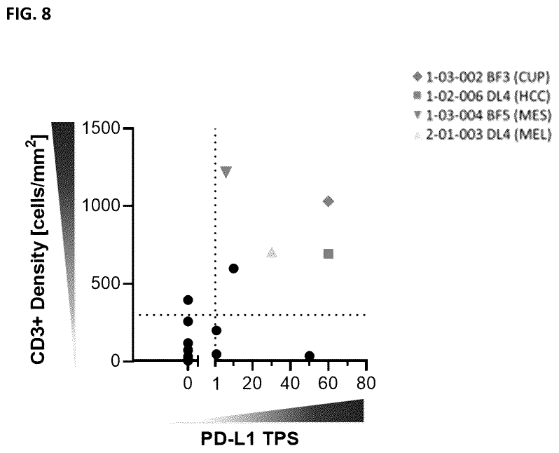

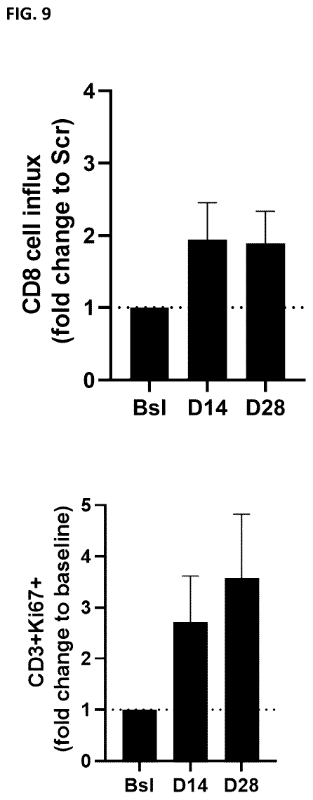

(FIG. 1A): Female albino C57Bl/6 mice were injected with 1×10⁶ Panc02-Luc cells into the tail of the pancreas. Animals were randomized by bioluminescence imaging on day 5, and treated twice-weekly with vehicle/anti-mGDF-15/anti-PD-1/anti-mGDF-15+anti-PD1. (FIG. 1B) and (FIG. 1C): Tumor load was assessed at least weekly via bioluminescent in vivo imaging. Tumor growth was assessed by relating the day 35 measurement to the signal during randomization. Tumor growth was compared by Mann-Whitney test and corrected for multiple comparisons according to Bonferroni-Holm.

FIG. 2: CTL-002 treated animals show higher immune cell infiltration enriched in CD3+ cells.

Syngeneic mouse models MBT-2 (left panel) and Pan02 (right panel) show higher immune cell infiltration in aGDF-15 treated animals.

FIG. 3: Sequence of the binding region of CTL-002 in various species

The sequences of the binding regions of CTL-002 are shown for humans (SEQ ID NO: 14), cynomolgus monkeys (SEQ ID NO: 15), mice (SEQ ID NO: 16) and rats (SEQ ID NO: 17) (first four lines from top to bottom).

FIG. 4: GDF-15 serum levels in female monkeys following the first dose of CTL-002

FIG. 5: Predicted GDF-15 suppression in the tumor micro-vasculature considering different dosing intervals

Predicted suppression of GDF-15 in the tumor micro-vasculature Assumption: three baseline levels of systemic GDF-15 (0.5, 2 and 10 ng/mL), three different CTL-002 concentrations (3, 10 and 20 mg/kg) and three different CTL-002 dosing intervals (2, 3 and 4 weeks)

FIG. 6: Visualisation of correlation between clinical response and PD-L1 TPS and TIS

Baseline biopsies from clinical responder 1-02-006, 1-03-004, 1-03-002 and long-term SD 2-01-003 show PD-L1 TPS >1 and Tumor Inflammation Score (TIS) >7.5, suggesting a CTL-002/Nivolumab response predictive value of PD-L1 TPS and TIS in anti-PD-1/PD-L1 refractory patients. Moreover, 6 out of 19 patients show PD-L1 TPS >1 and 50% (3 out of 6) thereof show PR.

FIG. 7A-FIG. 7B: Correlation of PD-L1 TPS versus CD8 cell counts

(FIG. 7A) Baseline biopsies from clinical responder 1-02-006, 1-03-004, 1-03-002 and long-term SD 2-01-003 show PD-L1 TPS >1 and CD8+ T cell density >300 cells/mm², suggesting a CTL-002/Nivolumab response predictive value of PD-L1 TPS and CD8+ T cell density in anti-PD-1/PD-L1 refractory patients. Moreover, 6 out of 19 patients show PD-L1 TPS >1 and 50% (3 out of 6) thereof show PR. In addition, 5 out of 19 patients show PD-L1 TPS >1 and CD8 cell density >300 cells/mm² and 60% (3 out of 5) thereof show PR.

(FIG. 7B) Updated version of (FIG. 7A) including additional patients including inter alia clinical responders 1-02-036 and 1-06-005 also show PD-L1 TPS >1 and CD8+ T cell density >300 cells/mm². Moreover, 27 out of 60 patients show PD-L1 TPS >1 and 18.5% (5 out of 27) thereof show PR. In addition, 15 out of 60 patients show PD-L1 TPS >1 and CD8 cell density >300 cells/mm² and 33.3% (5 out of 15) thereof show PR.

FIG. 8: Correlation of PD-L1 TPS versus CD3 cell counts

Baseline biopsies from clinical responder 1-02-006, 1-03-004, 1-03-002 and long-term SD 2-01-003 show PD-L1 TPS >1 and CD3+ T cell density >300 cells/mm², suggesting a CTL-002/Nivolumab response predictive value of PD-L1 TPS and CD3+ T cell density in anti-PD-1/PD-L1 refractory patients. Moreover, 6 out of 18 patients show PD-L1 TPS >1 and 50% (3 out of 6) thereof show PR.

FIG. 9: Fold change in CD8+ and CD3+ki67+ cells of treated human patients on days 14 and 28 compared to baseline (Bsl)

Immune cell analysis in biopsies from baseline (bsl), day 14 and day 28 of patients revealed an increase in CD8+ T cells (upper panel) and proliferating CD3+ki67+ T cells (lower panel) under treatment.

### DETAILED DESCRIPTION OF INVENTION

Unless specifically defined herein, all technical and scientific terms used herein have the same meaning as commonly understood by a skilled artisan in the fields of the invention.

All methods and materials similar or equivalent to those described herein can be used in the practice or testing of the present invention, with suitable methods and materials being described herein.

All literature references referred to herein are incorporated by reference in their entirety for all purposes. Patent applications are referred to herein by using their application and/or publication number. Non-patent literature referred to herein may be cited either as a full reference, or in abbreviated form (e.g., Danaher et al., 2018), followed by the full reference in the “References” section.

As used herein, each occurrence of terms such as “comprising” or “comprises” may optionally be substituted with “consisting of” or “consists of”.

The present invention provides an anti-GDF15 antibody which may be used in the treatment of cancer in human patients.

GDF-15 can be measured by ELISA. ELISAs which can be used to measure GDF-15 include but are not limited to the R&D systems Quantikine ELISA, an immunoradiometric assay, Luminex™ sandwich assay and electrochemiluminescence sandwich assay, as e.g. the ELECSYS® GDF15 assay (Roche Diagnostics), which was summarized by Wollert et al. (Wollert K C, Kempf T, Giannitsis E, et al. An Automated Assay for Growth Differentiation Factor 15. J Appl Lab Med An AACC Publ. 2018; 1(5):510-521. doi:10.1373/jalm.2016.022376). All mentioned assays are based on the immunosandwich principle using monoclonal or polyclonal antibodies to capture and to quantify the GDF-15. Dependent on the used reagents and their combination, free GDF-15 or total GDF-15 (free GDF-15 and GDF-15 bound to CTL-002) is measured.

As used herein the term “PD-L1 positive cancer” relates to a cancer which has been detected to be PD-L1 positive. It is understood that the term “PD-L1 positive cancer” means that the cancer is a PD-L1-positive cancer at the point in time when the anti-GDF-15 antibody is administered for the first time. In other words, the cancer is a PD-L1-positive cancer as can be assessed by analysis of a baseline biopsy from the human patient. This is to be distinguished from, but does not exclude, the additional possibility that a change of PD-L1 expression such as an upregulation of PD-L1 expression could take place during the course of administration of the anti-GDF-15 antibody. However, a “PD-L1 positive cancer” as referred to herein is to be distinguished from, and excludes, cancers which were not PD-L1-positive at the point in time when the anti-GDF-15 antibody is administered for the first time but became PD-L1-positive only subsequently due to an upregulation of PD-L1 expression during the course of administration of the anti-GDF-15 antibody. The detection method used for determining whether the cancer is PD-L1 positive is not particularly limited. Any method allowing to directly/indirectly detect the presence of PD-L1 on the cell surface of cells in the cancer may be used in the context of the present invention.

Direct methods for detecting PD-L1 may be based on assays for the detection of PD-L1 at the protein level. An exemplary method for the direct detection of PD-L1 uses immunohistochemistry to stain cells having PD-L1 on the cell surface. Indirect methods for detecting PD-L1 may be based on assays aiming to detect PD-L1 expression at the nucleotide level using e.g. gene expressing data. In a preferred embodiment of a direct method for detecting PD-L1, immunohistochemistry (IHC) assays is used to determine whether the cancer of a human patient is a PD-L1 positive cancer. The skilled person in the art is aware of IHC assays allowing to determine the fraction of PD-L1 positive cells within a cancer sample based on common general knowledge. In this respect, the skilled person is also aware that in order to meet the requirements of drug agencies (e.g. FDA) for the treatment using a checkpoint inhibitor, an immunohistochemical assay of the cancer tissue to detect the presence/fraction of PD-L1 positive cells is mandatory for some therapeutic agents. Hence, the skilled person using common general knowledge is readily aware of assays allowing to ascertain whether a cancer is positive for PD-L1. In general, IHC assays may be based on an anti-PD-L1 antibody as primary antibody allowing to bind to the PD-L1 protein located on the surface of cells in the cancer. Further analysis allows to determine the tumor proportion score (TPS) of the cancer, which is the percentage of cancer cells showing staining for the presence of PD-L1 on the cell surface. This percentage of cancer cells can be determined by counting, within the cancer sample, the number of all cancer cells showing staining for the presence of PD-L1 on the cell surface, and by dividing said number by the total number of cancer cells in the sample, and multiplying the resulting quotient by 100. Alternatively, a combined positive score (CPS) for PD-L1 can be determined, which is the summed-up percentage of cancer cells and cancer-infiltrating cells (e.g., cancer-infiltrating immune cells) showing staining for the presence of PD-L1 on the cell surface. This summed-up percentage of cancer cells and cancer-infiltrating cells can be determined by counting, within the cancer sample, the sum of the numbers of all cancer cells and cancer-infiltrating cells showing staining for the presence of PD-L1 on the cell surface, and by dividing said number by the sum of the total numbers of cancer cells and cancer-infiltrating cells in the sample, and multiplying the resulting quotient by 100. Preferably, a “PD-L1 positive cancer” has a percentage of cancer cells (also known as TPS) showing staining for the presence of PD-L1 on the cell surface of at least 1.

An exemplary method for determining the PD-L1 TPS is as follows:

For test interpretation and scoring, reference is made to the manufacturer's interpretation guideline: “VENTANA PD-L1 (SP263) Assay Staining of Non-Small Cell Lung Cancer Interpretation Guide”, version 1015317EN. Briefly, percentage of PD-L1 positive tumor cells (TPS) can be determined using an anti-PD-L1 monoclonal antibody assay such as the SP263 monoclonal antibody assay (Ventana, cat. #790-4905) in an immunohistochemistry (IHC) assay procedure based on formalin fixed and paraffin embedded (FFPE) sections. In anti-PD-L1 monoclonal antibody assays, tissue sections can be deparaffinized and heat pretreated prior to antibody incubation to uncover all target antigens. Next, anti-PD-L1 antibody binding to PD-L1 positive cancer cells is visualized using a secondary HRP enzyme-conjugated antibody (e.g., Ventana optiView Universal DAB Detection Kit, cat. #760-700). The specific antibody-enzyme complex is visualized based on a precipitating enzyme reaction product. For determining the PD-L1 TPS, the CE-IVD cleared predictive assay (741-4905) can be used. Preferably, a defined cut-off of >1% membranous stained tumor cells is used to distinguish between negative and positive cancer tissue (e.g., NSCLC tissue) specimens. Positive cases can be further subdivided by application of an additional ≥5% and ≥10% cut off. An exemplary slide interpretation and cut-off decisions can be performed as described in more detail in the official scoring guideline “VENTANA PD-L1 (SP263) Assay für Non-Small Cell Lung Cancer Interpretationsanleitung 2016”, version 1015317EN.

For scoring, the percentage of PD-L1 positive tumor cells was determined for each staining intensity (0, 1, 2, 3) and the total percentage of stained tumor cells was calculated by summing up the percentage at the intensity 1, 2 and 3.

As used herein the term “inflamed tumor” refers to an inflamed tumor phenotype. The skilled person is aware of assays to determine whether a tumor shows an inflamed tumor phenotype using common general knowledge.

As an exemplary method, the question of whether a tumor is an inflamed tumor may be assessed based on the gene expression analysis using a subset of genes related to antigen presentation, chemokine expression, cytotoxic activity, and adaptive immune resistance which translates into a tumor inflammation signature (TIS) of the tumor. The subset of genes used to determine the TIS is not particularly limited and may relate to at least one gene selected from the group consisting of PSMB10, HLA-DQA1, HLA-DRB1, CMKLR1, HLA-E, NKG7, CD8A, CCL5, CXCL9, CD27, CXCR6, IDO1, STAT1, TIGIT, LAG3, CD274, PDCD1LG2 and CD276. In a preferred embodiment, the subset of genes used to determine the TIS consists of all of the above-mentioned genes. The TIS score may be determined as a linear combination of the above-indicated 18 genes, calculating

$$T I S = {\sum }_{i = 1}^{18} {x}_{i} {w}_{i} ,$$

where xᵢ is the i^{th} gene's log 2-transformed, normalized expression (e.g., mRNA expression) level and wⱼ is a predefined weight above zero, e.g., as described in Ayers M, Lunceford J, Nebozhyn M, Murphy E, Loboda A, Kaufman D R, Albright A, Cheng J D, Kang S P, Shankaran V, Piha-Paul S A, Yearley J, Seiwert T Y, Ribas A, McClanahan T K. IFN-γ-related mRNA profile predicts clinical response to PD-1 blockade. J Clin Invest. 2017 Aug. 1; 127(8):2930-2940. doi: 10.1172/JCI91190. Epub 2017 Jun. 26. PMID: 28650338; PMCID: PMC5531419.

Alternatively or additionally, the question of whether a tumor is an inflamed tumor may be assessed by assessing whether the tumor is a tumor which contains CD8+ or CD3+ cells. Preferably, the inflamed tumor contains CD8+ or CD3+ cells as determinable by an immunohistochemistry (IHC) assay, wherein the IHC assay is preferably based on an anti-CD8 or anti CD3 antibody as primary antibody. More preferably, it can be assessed whether the inflamed tumor is a tumor having an average CD8+ or CD3+ cell density of >300 cells/mm² in immunohistochemical tissue sections having a thickness of, for instance, 4 μm, as assessed by the immunohistochemistry (IHC) assay.

An exemplary immunohistochemistry (IHC) assay for CD8+ cells is as follows: A primary (monoclonal) anti-CD8 antibody (e.g., Monoclonal Mouse Anti-Human CD8, clone C8/144B) can be applied in a multiplex IHC assay procedure to Formalin-Fixed Paraffin-Embedded (FFPE) cancer tissue sections (thickness: e.g., 4 μm). To uncover all target antigens, tissue sections are deparaffinized and heat pretreated prior to antibody incubation. Binding of the specific antibody (e.g., the anti-CD8 antibody) can be visualized using a secondary horseradish peroxidase (HRP)—or alkaline phosphatase (AP)—enzyme-conjugated antibody, for example, subsequent to application of one or more linker antibodies, hydroxyquinoxaline (HQ)—or nitropyrazole (NP)—conjugated, thus allowing for amplification of the resulting signal. The enzyme coupled to the secondary antibody catalyzes a chromogenic precipitate-forming reaction at the binding site of the primary antibody. Each step was incubated. After each incubation step, an automated slide stainer (e.g., a VENTANA DISCOVERY ULTRA automated slide stainer) may wash the sections to stop the respective reaction and to remove unbound material. Prior to application of the next primary antibody, if applicable, the previous reagents are removed by heat incubation (“stripping”), leaving only the insoluble precipitate on the slide. For quantitative evaluation of stained tissue sections, a suitable digital image analysis software (e.g., Visiopharm digital image analysis software VIS 2019.02 (TID 001673)) can be used. Stained slides may be scanned at a magnification of 20×, e.g., using an Aperio ScanScope XT (Leica), prior to image analysis. The final workflow allows detection of not only the positive staining for the single biomarkers (e.g., CD8) but is also able to discriminate between the tissue compartments of tumor, normal tissue and peri- and intratumoral stroma. To achieve this, an additional guidance staining with the tumor-specific markers panCK (solid tumors) or SOX10 (malignant melanoma) can be used, allowing for generation of virtual overlay images by alignment of consecutive sections and transfer of information from one slide to the other. The final readout can include area measurements (mm²) per tissue compartment, cell counts (e.g., CD8 cell counts) per cell type per compartment, density calculations (cell counts per mm²) and distance measurements for biologically relevant combinations of cell types.

CD3+ T cell density can be determined, for example, by an immunohistochemistry (IHC) assay using an anti-CD3 antibody, e.g., using a ULTIVUE® Multiplex Immunofluorescence Assay (UltiMapper I/O T-act Panel CD3, Granzyme B, Ki67 and a panCK/SOX10) in 4 μm thick FFPE tissue sections, using the UltiMapper® I/O T-act kit by ULTIVUE (p/n ULT20110) stained on Leica Bond RX, digitized as full-slide images using ZEISS Axioscan Z1 and subsequent digital analysis in Visiopharm.

As used herein the term “clinical outcome” has the normal meaning as understood by the skilled person in the art. The term “clinical outcome” refers to events or endpoints which are reached upon medical treatment. Classical measures to evaluate the efficiency of a treatment in clinical terms may be used to describe the clinical outcome. Exemplary measures reflecting a clinical outcome are overall survival (OS) and progression-free survival (PFS). Moreover, the clinical outcome may also include initial efficacy endpoints such as response rate, complete response, partial response, stable disease, survival or progressive disease.

In a preferred embodiment in accordance with all other embodiments of the invention, the cancer is a “solid cancer”. A “solid cancer” is a cancer which forms one or more solid tumors. Such solid cancers forming solid tumors are generally known in the art. The term “solid cancer” encompasses both a primary tumor formed by the cancer and possible secondary tumors, which are also known as metastases. “Solid cancers” encompass all non-hematologic cancers and include but are not limited to colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP) and breast carcinoma.

As referred to herein, the terms “CTL-002”, “CTL-001 IgG4*”, “CTL-001 IgG4” and “H1L5 IgG4*” and visugromab are used synonymously. They refer to an antibody having the heavy chain amino acid sequence of SEQ ID NO: 8 and the light chain amino acid sequence of SEQ ID NO: 9. It is understood that any reference to the amino acid sequences of antibodies of the invention is meant to encompass posttranslational modifications of these sequences occurring in mammalian cells such as CHO cells, including, but not limited to, N-glycosylation, O-glycosylation, deamidation, Asp isomerization/fragmentation, pyro-glutamate formation, removal of C-terminal lysine, and Met/Trp oxidation.

In a preferred embodiment in accordance with all other embodiments of the invention, the GDF-15 is human GDF-15 (also referred to herein has “hGDF-15”) and the anti-GDF-15 antibody is an anti-human GDF-15 antibody (also referred to herein as “anti-hGDF-15 antibody”).

It is understood that the antibodies according to the invention can be administered in the form of pharmaceutical compositions. Such pharmaceutical compositions are prepared in a way that they can be stored and administered appropriately, e.g. by using pharmaceutically acceptable components such as carriers, excipients or stabilizers.

Such pharmaceutically acceptable components are not toxic in the amounts used when administering the pharmaceutical composition to a human patient. The pharmaceutical acceptable components added to the pharmaceutical compositions may depend on the route of administration.

In general, the pharmaceutically acceptable components used in connection with the present invention are used in accordance with knowledge available in the art, e.g. from Remington's Pharmaceutical Sciences, Ed. AR Gennaro, 20th edition, 2000, Williams & Wilkins, PA, USA.

The invention is illustrated by the following non-limiting examples and reference examples.

### (1) Reference Examples

### Drug Substance (DS)

CTL-002 is a humanized, hinge-stabilized IgG4 monoclonal antibody targeting Growth Differentiation Factor-15 (GDF-15) and relates to an antibody of the present invention.

In an exemplified liquid formulation of the CTL-002 Drug Substance, the CTL-002 antibody is presented at a concentration of about 25 mg/mL, further comprising 20 mM Histidine/Histidine HCl, 150 mM sucrose, 50 mM Arginine-HCl and 0.02% w/v Polysorbate 20, at a pH of 5.5.

### Manufacturing & Control

The CTL-002 Drug Substance may be manufactured in CHO cells such as CHOK1SV GS KO™ cells. The downstream process includes 2 chromatography steps; one Protein A-based affinity affinity chromatography (e.g. MabSelect SuRe) followed by anion exchange membrane chromatography (e.g. Sartobind Q).

Virus inactivation is achieved by e.g. Triton-X 100 treatment.

Analytical testing is performed routinely in-process and for the final release.

### Stability

Drug Product stability studies are currently ongoing as follows:
Pilot non-cGMP (Batch #DPS026) representative stability study for (up to) 3 years for the intended long-term storage conditions at +2-8° C., and for 12 months at +25° C. and for 6 months at +40° C. in order to provide representative stability data for the setting of provisional shelf life of Drug Product GMP batch(es).
GMP (Batch #F19235) stability study for (up to) 3 years for the intended long-term storage conditions at +2-8° C., and for 12 months at +25° C. and for 6 months at +40° C. in order to confirm the stability of the proposed IMP.

### Storage

CTL-002 Drug Product vials must be stored at +2-8° C. in their original secondary packaging within a secure environment, protected from light and separated from other medication or investigational product. The product should not be frozen.

### Preparation & Administration

To prepare CTL-002 for intravenous administration, the CTL-002 solution is added to an infusion bag containing 0.9% NaCl. CTL-002 solution for infusion may be administered using IV bags made of polyethylene (PVC-, DEHP- and latex-free) or polyvinylchloride (latex-free) and infusion lines made of PE (PVC-, DEHP- and latex-free) or PVC (DEHP- and latex-free) material. The use of an 0.2 μm in-line filter (positive charged/uncharged PES membrane) is mandated.

Once compounded, the CTL-002 solution for infusion in infusion bags may be used immediately and administered at ambient temperature. The infusion bag might be stored up to 6 hours at room temperature and up to 24 hours at +2-8° C. but should be used no longer than 24 hours after preparation.

### Nonclinical Pharmacology

The present inventors identified a mechanism by which GDF-15 blocks adhesion and transgression of predominantly CD8+-T-lymphocytes into tissues. With CTL-002 blocking GDF-15 a novel treatment approach has been established that facilitates effector T cell entry into tumor tissue. This may substantially enhance the efficacy of any T cell activating agent, e.g. checkpoint inhibitors.

Specifically, in a flow-adhesion assay, different immune cell subsets pre-treated +/− GDF-15 were perfused over an activated layer of endothelial cells or recombinant adhesion molecules. Adhesion and transmigration processes were monitored by live imaging microscopy. Adhesion of T cells to the endothelial cell layer was significantly impaired by addition of GDF-15. Among T-cell subsets CD8+ T-cells were most affected while adhesion of other immune cells was not reduced. Inhibitory effects of GDF-15 on CD8+ T-cell adhesion were comparable to potent blockade of LFA-1 by TS1/18 antibody and could be rescued by the anti-GDF-15 antibody CTL-002 with an EC50 of ^{˜}700 ng/ml.

This initial finding has been further substantiated with data from relevant animal models, in which the anti-GDF-15 antibody CTL-002 or a mouse surrogate induced strong increase of tumor infiltrating lymphocyte numbers and increased the response to T cell activating therapies. Neutralization of GDF-15 in HV18-MK melanoma-bearing humanized mice by CTL-002 resulted in a strong increase of tumor infiltrating leukocyte numbers. Subset analysis revealed an over proportional enrichment of T-cells, especially CD8+-T cells (see FIG. 6 of WO 2022/101263). In line, GDF-15 neutralization in syngeneic mouse models MBT-2 and Pan02 led to a strong increase in T-cell infiltration (FIG. 2).

That GDF-15 can enhance response to checkpoint blockade was verified in orthotopic Pan02. Here combination of aGDF-15 with aPD-1 doubled the number of tumor-free animals over aPD-1 monotherapy (FIG. 1). Further, syngeneic mouse tumor models with genetically modified mouse colon tumor MC38 cells expressing human GDF-15 (MC38-GDF15), showed an increased response to otherwise diminished responses towards anti-PD-1 or anti-CD40/poly(IC:LC) combination therapy. No adverse effects were observed in any of the animals (FIG. 9-11 of WO 2022/101263).

Hence, CTL-002 is developed to neutralize the pathological effects mediated by GDF-15. The biological activity of GDF-15 adhesion and transmigration processes were monitored by live cell imaging microscopy in an in vitro flow adhesion system with primary immune cells and key parameter of the inhibition of GDF-15 effects by CTL-002 were determined in this system.

Moreover, secondary pharmacology studies examined the ability of CTL-002 to elicit CDC and ADCC, on-target/off-tissue binding as well as off-target binding. Safety pharmacology assessments were included in standard repeat-dose toxicity studies.

Overall, these studies provide a thorough characterization of the mechanism of action of CTL-002 as well as a well-supported rationale for its clinical examination in patients with cancer and GDF-15 elevation in the tumor microenvironment.

### Binding of the Drug CTL-002 to the Target GDF-15

As shown in Table 1, GDF-15 and CTL-002 form a main complex of two CTL-002 antibodies and two dimeric GDF-15 molecules in solution. Other complexes seem to be less favorable and only one additional complex of three CTL-002 and three GDF-15 was reliably detected. This complex was maximal during equimolar incubation of CTL-002 and GDF-15 but was still below 8% and decreased when the ratio was changed in either direction. Above three molar excess of the antibody, all GDF-15 is complexed by CTL-002 molecules and no tendency for formation of high molecular weight aggregates was observed.

TABLE 1
Overview of formed complexes of CTL-002 and GDF-15

| Simplified Visualization | 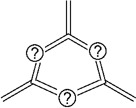 | 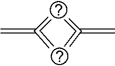 | 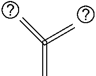 | 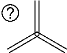 | 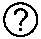 |
|---|---|---|---|---|---|
| Estim. MW | 506 kDa | 337 kDa | 169/193 kDa | 144 kDa | 24.6 kDa |
| Ratio CTL- | [CTL-002]₃- | [CTL-002]₂- | [CTL-002]₁- | CTL-002 | GDF-15 |
| 002/GDF-15 | [GDF-15]₃ | [GDF-15]₂ | [GDF-15]_{1/2} | | |
| 1:30 | 1.1% | 15.7% | 4.2% | 0% | 79.1% |
| 1:3 | 4.9% | 70.3% | 4.3% | 0% | 20.6% |
| 1:1 | 8.0% | 74.3% | 4.0% | 13.0% | 0.6% |
| 3:1 | 2.9% | 27.5% | 0% | 69.6% | 0% |
| 30:1 | 0.4% | 2.9% | 0% | 96.7% | 0% |

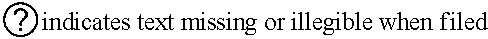

Affinity of CTL-002 to recombinant human GDF-15 was determined by measuring surface plasmon resonance on a Biacore T3000. In addition, the affinity to cynomolgus rat and mouse GDF-15 was measured (see Table 2).

All experiments were performed in 10 mM HEPES buffer, pH 7.4, 150 mM NaCl, 3.4 mM EDTA and 0.05% Tween. anti-human IgG Fc specific antibody (Jackson, Order #109-005-008, Lot #111148) was covalently immobilized by EDC/NHS chemistry on Biacore CM5 Chips (GE Healthcare, Order #61144275, Lot #10236645). For kinetic characterization of the antigen-antibody interaction pulses of increasing GDF-15 concentrations (e.g. 156.3 pM, 312 pM, 625 pM, 1,250 pM) were injected at a flow rate of 30 μl/min. After each measurement cycle (8 min of association followed by 30 min of dissociation) the antibody-antigen complex was resolved by regeneration of the surface with 10 mM glycine-HCl at pH 2.0. For calculation of the dissociation constant of CTL-002 the association and dissociation phases were recorded and evaluated by global fitting using the software BIAevaluation 4.1. For global fit analysis only, these antigen concentrations were taken into account, which allowed the analysis following the Langmuir 1:1 binding model or 1:1 binding with drifting baseline.

K_{D} values for human GDF-15 are shown in Table 2.

TABLE 2
Overview of species affinities of CTL-002

| | CTL-002 | |
|---|---|---|
| K_{D} (human) | 38.3 | pM |
| K_{D} (Cynomolgus) | 107.7 | pM |
| K_{D} (rat) | 449 | pM |
| K_{D} (mouse) | 9.76 | nM |

An overview and comparison of the three antibodies used in a panel of non-clinical studies is given in Table 3.

TABLE 3
Overview on antibody details used in in vitro and in vivo experiments

| | Anti-mGDF-15 | B1-23 | CTL-001 | CTL-002 |
|---|---|---|---|---|
| Binding domain | Not known | Discontinuous epitope (EVQVTMCIGACPSQFR (SEQ ID | | |
| | | NO: 12) ---38 amino acids ---TDTGVSLQTYDDLLAKDCHCI | | |
| | | (SEQ ID NO: 13)) | | |
| Species | mouse | mouse | humanized | humanized |
| Isotype | IgG1 | IgG2a (rec IgG1) | IgG1 | IgG4S228P |
| Alternative | | | H1L5, B1-23- | CTL-001 IgG4*, |
| names | | | H1L5 | CTL-001 IgG4, |
| | | | | H1L5 IgG4* |
| Characterization | Efficacy studies | Epitope | | |
| studies | in syngeneic | mapping by | | |
| | mouse models | proteolytic | | |
| | MBT-2 and | excision | | |
| | Pan02 | | | |
| | | ADCC, C1q and | ADCC, C1q | ADCC, C1q |
| | | CDC | binding and | binding and CDC |
| | | | CDC | |
| | | Immunoblot and | Immunoblot | ELISA of human |
| | | ELISA of human | and ELISA of | GDF-15 |
| | | GDF-15 | human and | |
| | | | mouse GDF-15 | |
| | | Flow cytometry | Flow cytometry | |
| | | stain of surface | stain of surface | |
| | | associated GDF- | associated GDF- | |
| | | 15 | 15 | |
| | | | Immunoblot | |
| | | | and ELISA of | |
| | | | different TGF- | |
| | | | beta proteins | |
| | | | Inhibition of | |
| | | | LFA-1 activation | |
| | | | by D-Storm | |
| | | | microscopy | |
| | | | Inhibition of | |
| | | | adhesion of | |
| | | | flowing T cells | |
| | | | to preactivated | |
| | | | HUVECs | |
| | | In vivo | | |
| | | combination | | |
| | | with anti- PD-1 | | |
| | | to treat | | |
| | | transgenic | | |
| | | MC38 secreting | | |
| | | hGDF-15 in mice | | |
| | | In vivo | | |
| | | combination | | |
| | | with anti- | | |
| | | CD40/Poly(IC:LC) | | |
| | | to treat | | |
| | | transgenic | | |
| | | MC38 tumors | | |
| | | secreting hGDF- | | |
| | | 15 in mice | | |
| | | In vivo | In vivo | |
| | | neutralization of | neutralization | |
| | | cachexia in mice | of cachexia in | |
| | | | mice | |
| | | | In vivo | In vivo preliminary |
| | | | preliminary PK | PK study in |
| | | | study in | cynomolgus |
| | | | cynomolgus | monkeys |
| | | | monkeys | |
| | | | | In vivo treatment |
| | | | | with CTL-002 of |
| | | | | humanized mouse |
| | | | | human melanoma |
| | | | | model |
| | | | | Non-GLP single- |
| | | | | dose dose- finding |
| | | | | study in |
| | | | | Cynomolgus |
| | | | | monkeys |
| | | | | PK and Dose- |
| | | | | range finding in |
| | | | | rats |
| | | | | Mouse PK |
| | | | | 4-Week Repeat- |
| | | | | Dose Toxicity |
| | | | | Study of CTL-002 |
| | | | | in Cynomolgus |
| | | | | Monkeys |

In summary, CTL-002 is a humanized IgG4 antibody derived from mouse antibody B1-23 with high specificity for human GDF-15. CTL-002 binds with picomolar affinity to human, cynomolgus and rat GDF-15 (38.3, 108 and 449 pM, respectively), and with low nanomolar affinity to mouse GDF-15 (9.76 nM). CTL-002 binds to a discontinuous conformational epitope at the carboxy terminus of the mature GDF-15 and specifically recognizes the physiological dimeric conformation.

### In Vitro Biological Activity of CTL-002 on GDF-15 Mediated Inhibition of T Cell Adhesion

A flow adhesion assay system was used to mimic the dynamics at the blood vessel wall separating the immune cells from the tumor tissue and to analyze the effect of neutralization of GDF-15 by CTL-002. To evaluate the potency of GDF-15 adhesion inhibition and the sensitivity of the system, the IC50 of GDF-15 in the assay system was determined to 13.8±2.3 ng/ml. This was shown in FIG. 4 of WO 2022/101263.

To evaluate the potency of CTL-002 in preventing GDF-15 mediated inhibition of T cell adhesion, the EC50 of CTL-002 was determined in the flow-adhesion assay with T cells. As a result, in average a concentration of 707±17 ng/ml of CTL-002 was effective to increase the T cell adhesion by 50%, as was shown in different donors. This was shown in FIG. 5 of WO 2022/101263.

### In Vivo Pharmacology

Among all TGF-beta superfamily members orthologous GDF-15 molecules show the lowest sequence conservation. While mature rat, mouse and human TGF-betal and BMP-2 proteins are 99-100% sequence identical between humans and mice, homology is below 70% for GDF-15 (Böttner 1999). Biological differences between different species thus cannot be ruled out. Murine GDF-15 shows rather low sequence identity of 67.9% with its human counterpart reflected also in the fact that the anti-GDF-15 antibodies show less affinity for the mouse homologue. Two different in vivo approaches were followed to investigate pharmacodynamic effects.

First, a humanized mouse model was used, where immunodeficient mice are engrafted with human cord-blood derived CD34+-hematopoietic stem cells.

Three months after reconstitution these mice developed a functional human-like immune system containing all major human immune cell subsets (Wang 2018). These mice were then inoculated with a patient-derived human melanoma cell line, HV-18MK, that has been shown to secrete high levels of GDF-15, was inoculated. Mice were treated beginning three days later with isotype control or CTL-002 two times a week and after four weeks tumors were harvested and analyzed for immune cell infiltration by flow-cytometry.

Although the tumor size was unaffected, the CTL-002 treated groups showed a 9-fold increase in human tumor infiltrating CD45+ cells. This was shown in FIG. 6 of WO 2022/101263. Within the infiltrating cell population, T-cells were enriched 4-fold. In a follow-up study, analysing the infiltrating immune cell population in more detail, the increase in infiltration of CD45+ and the enrichment in CD3+ was confirmed, albeit less pronounced with 3-fold increase of CD45+ and 4-fold enrichment of CD3+ T cells. Similarly, syngeneic mouse models MBT-2 (FIG. 2, left panel) and Pan02 (FIG. 2, right panel) show higher immune cell infiltration in aGDF-15 treated animals.

As a second experimental model, a genetically modified mouse cell line overexpressing human GDF-15 was generated for testing the therapeutic efficacy of anti-GDF-15 antibodies.

In this respect, MC38 colon adenocarcinoma cells are the preferred mouse tumor cells to analyze immune checkpoint blocker activity (Selby 2016) and were used to generate in vivo data to support the development of the approved anti-PD-1 antibody. Overexpression was implemented by stable transfection and did not affect in vitro proliferation compared to control treated MC38.

Age matched immunocompetent C57BL/6 and immunodeficient NCI nu/nu mice were inoculated subcutaneously with a suspension of 5×10⁵ MC38 blank colon tumor cells or the transgenic derivative MC38^{GDF-15} expressing recombinant human GDF-15. In contrast to in vitro proliferation and in vivo proliferation in immunodeficient NCI nu/nu mice, it was previously shown that GDF-15 expression mediated a growth advantage in immunocompetent C57BL/6 mice supporting the idea that GDF-15 interferes with the immune system of the tumor host (see FIG. 7 of WO 2022/101263).

Further, expression of human GDF-15 rendered anti-PD-1 responsive MC38 colon carcinoma tumors into anti-PD-1 resistant tumors. This was partially reversed by anti-GDF-15 (B1-23)/anti-PD-1 combination treatment, but not by anti-GDF-15 monotherapy and partially by anti-PD-1 alone.

Similarly, monotherapy using an anti-GDF-15 antibody showed only minimal improvement, whereas GDF-15 secreting tumors (solid line, FIG. 9B of WO 2022/101263) showed better survival when treated with a combination of anti-GDF-15 B1-23 and anti-PD-1 (FIG. 9 of WO 2022/101263). This combination treatment was non-significantly better than anti-PD-1 mono therapy (FIG. 8 of WO 2022/101263).

Since CTL-002 and its derivatives have only nanomolar affinity for mouse GDF-15, a surrogate antibody anti-mGDF-15 (having light chain variable region domain amino acid sequence of SEQ ID NO: 19 and a heavy chain variable region domain amino acid sequence of SEQ ID NO: 20) with picomolar affinity for mouse GDF-15 was used for another syngeneic mouse model. GDF-15 secreting murine pancreatic adenocarcinoma Panc02 cells, expressing a luciferase gene for monitoring tumor growth, were implanted into the mouse pancreas. The implantation of the cell line into the corresponding tissue allows to assay tumor development in a relevant environment that mimics the disease process in humans more closely. After 5 days mice were randomized into four groups and treated two times a week with isotype antibody, anti-PD-1, anti-GDF-15 or anti-PD-1 or a combination of anti-GDF-15 and anti-PD-1. The tumor size was measured regularly by luciferase imaging. The relative results of day 35 are summarized in FIG. 1 in a waterfall plot ranking tumor development of each animal within a group from base line and a bar graph showing the relative mean tumor size change from baseline (FIG. 1). In contrast to the vehicle group where one of twelve animals showed spontaneous tumor shrinkage for unknown reason, tumor shrinkage was only observed for five of twelve animals in the combination treatment group and to a lower extent in three of twelve animals in anti-PD-1 monotherapy group (FIG. 1).

Since anti-GDF-15 treatment is suggested to increase T cell infiltration, other cancer immunotherapies depending on T cell presence in the tumor should benefit from neutralization of GDF-15.

Another immunotherapy, anti-CD40 and poly(IC:LC) tumor treatment, which was shown previously to depend on T cell immune responses (van den Boorn 2013) was previously tested in a mouse model with MC38 cells expressing human GDF-15. The combination treatment using GDF-15 neutralization and anti-CD40/poly(IC:LC) resulted in complete rejection of the tumors in 8 out of 10 animals, whereas only 3 out of 10 tumors were cleared by anti-CD40/poly(IC:LC) treatment alone (FIG. 10 of WO 2022/101263).

Although mouse GDF-15 could not be detected in wildtype MC38 cells, which might be due to insufficient analytic assays, a similar experiment comparing isotype control antibody with anti-GDF-15 treatment in combination with anti-CD40/poly(IC:LC) was done with MC38 cells that were not manipulated to secrete human GDF-15. Similar to the experiment with genetically modified MC38, anti-GDF-15 could improve efficacy of the anti-CD40/poly(IC:LC) treatment, however to a lesser extent (FIG. 11 of WO 2022/101263).

In the NHP 4wk GLP toxicity study, CTL-002 has been shown to be safe and well tolerated at doses which provide adequate exposure margins for clinical testing.

The proposed FIH starting dose of 0.3 mg/kg/Q2wk is projected to give a maximum plasma concentration (Cmax) at the end of the 1 hour infusion of 6 μg/mL, which is 683-fold lower than Cmax exposure to CTL-002 at the No-Observed-Effect-Level (NOEL) in NHP. This dose may achieve only transient suppression of GDF-15 in the tumor micro-environment and is considered to be a minimal acceptable biological effect level (MABEL).

Serum total GDF-15 has been shown to be a useful biomarker of GDF-15 target engagement in NHP and CTL-002 doses >10 mg/kg are associated with maintenance of GDF-15 capture (and by inference, GDF-15 suppression). Hence, the total human GDF-15 may be a potential clinical biomarker of GDF-15 target engagement and the FIH clinical study is designed to explore both maximum suppression of GDF-15 for a limited time period and continuous suppression of GDF-15 throughout each dosing cycle.

### Toxicology

CTL-002 is being tested for the treatment of patients with advanced cancer.

To identify relevant species for non-clinical testing, sequence homology of GDF-15 was compared across species. Sequence homology from humans to Cynomolgus monkeys, mice and rats is 94.6%, 67.9% and 66.1%, respectively.

CTL-002 binds to a non-linear conformational epitope within GDF-15, illustrated as two boxes in FIG. 3. The sequences of the binding regions of CTL-002 are shown for Cynomolgus monkeys, humans, mice and rats (first four lines from top to bottom).

The cynomolgus monkey displays 100% sequence homology with the human CTL-002 binding epitope within GDF-15. It is therefore regarded as relevant species for toxicity testing. The binding affinity of CTL-002 to human and cynomolgus GDF-15 is 38.3 pM and 108 pM, respectively.

A dose response finding (DRF) study of CTL-002 has been performed in rats, as the product was expected to be sufficiently pharmacologically active to achieve sustained complete target inhibition at reasonable intravenous dose levels (binding affinity to rat GDF-15: 449 pM). However, PK/PD data obtained in the study indicated that CTL-002, even at the highest dose level 100 mg/kg, was not able to saturate GDF-15 binding. Therefore, pivotal toxicology was only assessed in the Cynomolgus monkey in a 4-week study with once weekly intravenous administration of CTL-002. No toxicity was observed up to the highest tested dose of 100 mg/kg.

A Good Laboratory Practice (GLP)-compliant tissue cross-reactivity study was conducted using human and Cynomolgus monkey tissues. Staining with CTL-002 in the tissue panels examined was limited to the cytoplasm of trophoblasts in the human and monkey placenta, which was consistent with the reported expression of its target protein, GDF-15, in the placenta. No unanticipated cross-reactivity was observed.

### Toxicology: Non-Pivotal Studies

A non-GLP single-dose dose-finding study in Cynomolgus monkeys was performed. The main objective of this study was to support dose selection for the subsequent GLP-compliant 28-day repeat-dose toxicity study. Furthermore, the pharmacokinetics of CTL-002 at various dose levels were characterized.

One male and one female animal, per dose level (0.1; 1; 10 or 100 mg/kg) were dosed by a single 30-minute intravenous infusion and pharmacokinetic profiles were recorded over 14 weeks. In addition to the pharmacokinetics analysis the occurrence of anti-drug antibodies was assessed pre-dose, as well as 6 and 12 weeks after dosing. GDF-15 and CTL-002 serum levels were quantified, pharmacokinetic data were calculated as total CTL-002 (PK total) and as free CTL-002 (PK free), separately.

As indicators of toxicity, mortality and clinical signs were checked daily. Body weights and food and water consumption were analyzed weekly and body temperatures, ECGs, blood pressure, hematology incl. coagulation as well as clinical chemistry including cytokines were assessed on several occasions during the study.

None of the animals died prematurely, and there were no CTL-002-related signs of toxicity at any of the tested dose levels.

The single infusion of 0.1, 1, 10 or 100 mg/kg CTL-002 on test day 1 led to a dose-related increase of the total GDF-15 serum level in all males and females. Target saturation by CTL-002 at dose levels of 10 and 100 mg/kg was indicated by overlapping GDF-15 curves at these two dose levels.

The measurement of total and free CTL-002 in serum samples obtained up to test day 43 (groups 1 and 2; treatment with 0.1 or 1 mg/kg CTL-002) or test day 99 (groups 3 and 4; treatment with 10 or 100 mg/kg CTL-002) revealed a dose-related exposure of the animals to CTL-002.

Key pharmacokinetic data are given in Table 4 as means of the one male and one female animal per group.

TABLE 4
Key pharmacokinetic data obtained in the monkey DRF study CTL-002-TOX-01

| | | | | | |
|---|---|---|---|---|---|
| | Dose level | | | | |
| Group | [mg/kg b.w.] | Cₘₐₓ [μg/ml] | t_{1/2} [h] | AUC₀₋ₜ [μgh/ml] | AUC_{inf} [μgh/ml] |

Total CTL-002

| | | | | | |
|---|---|---|---|---|---|
| 1 | 0.1 | 0.92 | 383.4 | 344.8 | 412.5 |
| 2 | 1 | 21.6 | 230.3 | 4631 | 4855 |
| 3 | 10 | 235.8 | 275.7 | 77692 | 78661 |
| 4 | 100 | 2751 | 352.6 | 666861 | 673354 |

Based on the results of this study, dose levels of 10, 30 and 100 mg/kg were recommended for the pivotal 4-week repeat dose toxicity study.

### Toxicology: Pivotal Studies

The additional pivotal study was a 4-week repeat-dose toxicity study of CTL-002 in Cynomolgus monkeys (age: 3-4 years) with a 4-week recovery period.

In this study, CTL-002 was administered by 30-minute intravenous infusions once weekly, i.e. on test days 1, 8, 15, 22 and 29. The recovery period ended on day 58. Dose levels of 0; 10; 30 or 100 mg/kg were administered to 3 male and 3 female monkeys per group plus 2 male and 2 female recovery animals in the control and high dose groups.

None of the animals died or had to be sacrificed prematurely and no test item-related signs of local intolerance were noted.

No test item-related effects were noted on behavior and external appearance, the body weight and body weight gain, the food and drinking water consumption, the electrocardiographic parameters and the heart rate, the circulatory functions, the hematological including coagulation parameters, the D-Dimer levels, the clinical chemistry parameters, the cytokine levels, the urinary parameters, the ophthalmological and auditory functions, the organ weights, and the myeloid:erythroid ratio of any of the animals at any dose level. No macroscopic organ changes were noted in any of the animals examined at any tested dose level.

Histopathology did not reveal any test item-related local or systemic lesions. No test item-related changes were noted during or at the end of the 4-week treatment-free recovery period.

Based on the above results, the No-Observed-Effect-Level (NOEL) was 100 mg CTL-002/kg by once weekly repeated intravenous 30-minute infusion for 4 weeks, i.e. 5 administrations per animal.

The Cmax-levels and AUC-areas for Total CTL-002 revealed a roughly linear dose-related systemic exposure of the animals. No sex-specific differences were noted. An accumulation of Total CTL-002 with time was noted with an accumulation ranging from approx. 2-fold to 6-fold. The calculated mean terminal serum elimination half-lives (t_{1/2}) of Total CTL-002 ranged from 119 to 651 hours.

Key pharmacokinetic data following the first (days 1-8) and fourth (days 22-29) administration of CTL-002 are given in Table 5 for the male and female animals.

TABLE 5
Key pharmacokinetic data for Total CTL-002 obtained in the monkey GLP
toxicity study CTL-002-TOX-03 after the first and fourth administration

| | | | | | |
|---|---|---|---|---|---|
| | Dose level | | | | |
| Group | [mg/kg b.w.] | Cₘₐₓ [μg/ml] | t_{1/2} [h] | AUC₀₋ₜ [μgh/ml] | AUC_{inf} [μgh/ml] |

Days 1-8

| | | | | | |
|---|---|---|---|---|---|
| 2 | 10 | M: 237 | M: 197 | M: 21500 | M: 47505 |
| | | F: 686 | F: 165 | F: 36901 | F: 67303 |
| 3 | 30 | M: 804 | M: 292 | M: 65026 | M: 193062 |
| | | F: 2292 | F: 212 | F: 103114 | F: 233692 |
| 4 | 100 | M: 2725 | M: 312 | M: 227335 | M: 717624 |
| | | F: 5428 | F: 458 | F: 533272 | F: 2981268 |

Days 22-29

| | | | | | |
|---|---|---|---|---|---|
| 2 | 10 | M: 1520 | M: 486 | M: 135788 | M: 503693 |
| | | F: 1030 | F: 119 | F: 89266 | F: 149202 |
| 3 | 30 | M: 4363 | M: 308 | M: 149037 | M: 450025 |
| | | F: 4935 | F: 169 | F: 360184 | F: 697479 |
| 4 | 100 | M: 12553 | M: 651 | M: 1388031 | M: 9257432 |
| | | F: 13624 | F: 234 | F: 1329769 | F: 3233365 |

Following infusion of CTL-002, GDF-15 serum levels of all animals at all dose levels increased up to 100-1000-fold and remained increased throughout the dosing interval. As indicated in FIG. 4, showing as example the GDF-15 levels in female monkeys throughout the first week after dosing, the increase was comparable across dose groups.

Therefore, it can be concluded that full target inhibition was obtained at all dose levels and throughout the dosing interval in the pivotal monkey study.

### Overall Study Design

A Phase 1 multi-center, first-in-human (FIH), open-label study consisting of Part A (dose escalation) followed by Part B (expansion) will be performed using the CTL-002 antibody. The main intent of the study is (a) to demonstrate safety of CTL-002 and the combination of CTL-002+anti-PD1/PD-L1 and (b) to demonstrate that patients relapsed post/refractory to anti-PD1/PD-L1 therapy due to elevated GDF-15 will respond again and show tumor shrinkage when the combination of CTL-002+anti-PD1/PD-L1 is administered.

### Part A (Dose Escalation)

At least 21 subjects will receive in “3+3” cohorts escalating doses of CTL-002 IV given as monotherapy and in combination with an anti-PD-1 checkpoint inhibitor in subjects with advanced-stage solid tumors that relapsed post or were refractory to a prior anti-PD-1/PD-L1 therapy and have exhausted all available approved standard treatments or are not eligible for them anymore. “Backfill cohorts” will recruit additional patients to the highest dose levels.

### Part B (Expansion)

Comprised of up to 8 expansion cohorts of up to 25 subjects per cohort in defined tumor entities that relapsed post or were refractory to a prior anti-PD-1/PD-L1 therapy to further evaluate the safety and efficacy of CTL-002 as monotherapy (one monotherapy cohort to be explored) or in combination with an anti-PD-1 checkpoint inhibitor (up to 8 combination cohorts to be explored) and to confirm the RP2D. The dedicated monotherapy cohort will serve to establish the safety profile of CTL-002 in prolonged monotherapy at a dose considered to be therapeutic (no anti-PD-1/PD-L1 added). Enrollment into all 8 cohorts may occur in parallel.

### Treatment Period

### Part A (Dose Escalation)

This study will employ a standard “3+3” dose escalation design for which 3 to 6 subjects will be enrolled at each assigned dose level, per cohort, depending on the occurrence of DLTs.

The planned doses of CTL-002 to be tested are outlined below:
Cohort 1: 0.3 mg/kg
Cohort 2: 1.0 mg/kg
Cohort 3: 3.0 mg/kg
Cohort 4: 10 mg/kg
Cohort 5: 20 mg/kg

The start dose of 0.3 mg/kg for Cohort 1 is fixed. Doses explored in Cohorts 2-5 (as outlined above) may be modified by the Safety Review Committee (SRC) based on emerging data (i.e., available safety, PK/pharmacodynamic, other biomarker data).

The DLT Observation Period will be the first two treatment cycles (i.e., first 4 weeks) for each dosing cohort. All treatment cycles are defined initially as 2 weeks in duration. CTL-002 will be administered once every two weeks as an IV infusion. Subjects will first receive one dose of CTL-002 given as monotherapy for one cycle, followed by a combination of CTL-002 given together with the defined checkpoint inhibitor for one cycle, where the defined checkpoint inhibitor will be administered at a dose of 240 mg IV given once every 2 weeks.

For the combination, CTL-002 and the defined checkpoint inhibitor will be given on the same day concomitantly, where CTL-002 will be administered first and for the first combination infusion, there will be a 30-minute observation period to assess safety, which will then be followed by the defined checkpoint inhibitor infusion. The period of observation may be modified (i.e., shortened or lengthened) based on emerging safety data.

The first two treatment cycles (i.e., first 4 weeks) represent the DLT Observation Period.

Thereafter, subjects will continue with the combination treatment, until progression or until withdrawal from the study for any other reasons (e.g., toxicity or subject withdraws consent).

Additional, intermediate dose cohorts may be explored based on emerging data and upon Safety Review Committee (SRC) request. The maximum dose of CTL-002 to be tested in this study will not exceed 20 mg/kg.

All subjects will be hospitalized overnight after receiving the first dose of CTL-002 and also after receiving the first combination dose of CTL-002 and the defined checkpoint inhibitor, for the purposes of safety observation and to enable logistical collection of sampling time-points (e.g., PK).

### Intra-Patient Dose Escalation in Extended Treatment

If any Cohort 1 subjects are still on 0.3 mg/kg treatment when Cohort 2 has been completed and reviewed by the SRC, the subjects can be increased to the Cohort 2 dose of 1.0 mg/kg.

If any Cohort 1 or 2 subjects are still on 1.0 mg/kg treatment when Cohort 3 has been completed and reviewed by the SRC, the subjects can be increased to the Cohort 3 dose of 3.0 mg/kg.

Note: Any subjects still on 0.3 mg/kg treatment must be treated at 1.0 mg/kg prior to advancing to 3.0 mg/kg in agreement with the Sponsor Medical Monitor.

The maximum a subject dose can be increased to, through intra-dose escalation, will be 3.0 mg/kg.

Available safety, PK/pharmacodynamic data, as well as preliminary efficacy data will inform the decision regarding the MTD/dose(s) to be further explored in Part B of the study.

The MTD is defined as the highest dose level of CTL-002 at which no more than 1 out of 6 subjects experienced a DLT during the first 2 treatment cycles (i.e., the first 4 weeks, where CTL-002 is given as monotherapy [Weeks 1 and 2] and in combination with the defined checkpoint inhibitor [Weeks 3 and 4]).

In addition, for Cohorts 3-5, in the absence of any DLT, an additional 3 subjects can be recruited into each of these cohorts (up to a total of 6 subjects per cohort), to increase the understanding of the PK and pharmacodynamic data. This occurs while dose escalation continues. These additional “backfill” subjects will receive the combination treatment of CTL-002 and the defined checkpoint inhibitor once every two weeks from Cycle 1 Day 1 onwards, with CTL-002 always administered first and the defined checkpoint inhibitor given thereafter as outlined above.

For Part A subjects (except backfill subjects), three sequential tumor biopsies are to be taken; one biopsy at baseline, the second biopsy prior to the initiation of the combination therapy (after 2 weeks) and the third after the first cycle of combination therapy (either at the End of Treatment or, if combination treatment is continued, at the end of Cycle 2/beginning of Cycle 3).

For backfill patients, only two biopsies are mandated; one at baseline, and the second after 4 weeks of combination treatment (either at the End of Treatment or, if combination treatment is continued, at the end of Cycle 2/beginning of Cycle 3).

These biopsies are mandatory in order to assess immune cell infiltration in the tumor. If a biopsy cannot be taken for safety reasons, this must be discussed with the medical monitor.

Treatment with CTL-002 in monotherapy (one cycle) as well as in combination with a checkpoint inhibitor (nivolumab) has been safely tolerated up to dose level 5 of CTL-002 (20 mg/kg). No DLT occurred, no grade 4 adverse event in any patient treated. Combination treatment can be started simultaneously, as demonstrated by the so-called backfill cohorts with immediate combination of CTL-002 and anti-PD1/PD-L1 treatment.

In line with the preclinical data on increase in immune infiltration by aGDF-15 biomarker analyses show a tumor selective influx of CD8+ cells from DL1-5 consistently. Preliminary analysis indicate that in several patients T cell proliferation is increased in the tumor as demonstrated by CD3/ki-67+ staining. Several tumors that at baseline are “cold tumors” characterized by rather low CD8 and CD4 counts are turned into hot tumors by increasing CD8 and CD4 counts.

Tumor shrinkages have been observed at various dose levels. Data are available for 24 evaluable subjects (Part A: 24/Part B: 0). All subjects were treated in a last line, heavily pretreated setting (on average 4.4 prior lines of therapy) and relapsed/refractory to prior anti-PD-1/PD-L1 treatment.

For 6 subjects in this heavily pre-treated last-line tumor population apparent and lasting clinical benefit was observed. Three subjects experienced a confirmed partial response (PR) currently up to and beyond 10 months duration (1× cancer of unknown primary on dose level [DL]3 and 1×mesothelioma on DL 5), the third subject experienced a confirmed PR with lasting, prolonged tumor regression (hepatocellular carcinoma on DL4). Three additional subjects experienced prolonged disease stabilization, 2 of them beyond 6 months (1×NSCLC and 2×melanoma). The third subject had 5.5 months of disease stabilization, then had local progression in one liver lesion compressing a vessel. Upon investigator decision study treatment was continued and local irradiation to the site added (off-study). Soon thereafter, the subject went on to a deep, confirmed partial response with abscopal effects seen in non-irradiated lesions (under continued study treatment with CTL-002 and nivolumab with permission of the sponsor). Two other subjects had minor tumor regression in isolated lesions.

In summary, in the dose escalation (Part A), 6 subjects have been treated for up to 10 months in the optional extended treatment phase and 2 even beyond one year and have shown lasting tumor control, including lasting, confirmed partial responses.

### Part B (Expansion)

In Part B of the study, up to 8 cohorts (up to 25 subjects per cohort) each enrolling subjects with a specific tumor type may be enrolled.

A dedicated CTL-002 monotherapy cohort may be set up in this expansion part of the study to explore the safety profile of CTL-002 given as monotherapy (e.g., in subjects with advanced-stage melanoma). In addition, for this monotherapy cohort, mandatory sequential tumor biopsies are required to broaden the understanding of the pharmacodynamic effects of CTL-002 in tumor tissue.

In this monotherapy cohort, two serial tumor biopsies are mandated: one biopsy to be taken at baseline and a second biopsy to be taken after 2 weeks (at the end of Cycle 1/beginning of Cycle 2). All subjects will be treated until progression.

Then, up to 8 other expansion cohorts of defined tumor populations may be treated with a combination of CTL-002 and the defined checkpoint inhibitor. Tumor indications will consist of PD-1/PD-L1 treatment approved tumor types and subjects that relapsed/progressed on or after anti-PD-1/PD-L1 treatment. Enrollment into expansion cohorts may occur in parallel. All subjects will be treated until progression.

For the purposes of safety observation and to enable logistical collection of sampling time points (i.e., PK sampling), all subjects will be hospitalized overnight after receiving the first dose of CTL-002 (monotherapy cohort) or after receiving the first combination dose with CTL-002 and the defined checkpoint inhibitor (combination therapy cohort), respectively.

### Study Assessments

### Tumor Biopsy

Timing of biopsies are as follows:
Part A (all subjects except backfill subjects): three sequential tumor biopsies are mandated; one biopsy at baseline, the second biopsy prior to the initiation of the combination therapy (after 2 weeks) and the third biopsy after the first cycle of combination treatment (either at the End of Treatment Visit or, if combination treatment is continued, at the end of Cycle 2/beginning of Cycle 3). These biopsies are mandatory in order to assess immune cell infiltration in the tumor.
Part A (backfill subjects): two serial tumor biopsies are mandated; one biopsy at baseline and a second biopsy after 4 weeks (either at the End of Treatment Visit or, if combination treatment is continued, at the end of Cycle 2/beginning of Cycle 3)
Part B (subjects enrolled in the monotherapy cohort): two serial tumor biopsies are mandated; one biopsy at baseline and a second biopsy after the first monotherapy cycle (i.e., 2 weeks).

There has to be a lesion that is amenable to sequential biopsy, if possible, or a lesion in close proximity, but this lesion should not be the only target lesion that will be radiologically assessed during the course of the study.

Biomarkers may be analyzed from biopsy tumor tissue samples. Additional immune cell markers and/or tumor markers specific to any of the tumor type may be included.

Biopsied tumor tissue will be fixed with formalin and embedded in paraffin (FFPE) to determine treatment-induced changes in the number, frequency and spatial location of infiltrating immune cells including but not limited to leukocytes, different lymphocytes (e.g., CD4+ and CD8+ T cells, B cells, NK cells) by histology before and after treatment with CTL-002 or in combination with the defined checkpoint inhibitor. Moreover, the expression of the CTL-002 drug target, GDF-15 protein and mRNA, will be determined.

### Tumor Lesions

For subjects in Part A of the study, the target cutaneous lesions selected for RECIST evaluation will be measured by caliper and photographed. In addition, the number of cutaneous lesions will be recorded. For clinical measurements of cutaneous lesions in Part A, documentation by color photography (including size measurement) and caliper measurement of lesion will be performed at Baseline within −7 days before infusion of CTL-002, every 4 weeks during extended treatment, at End of Treatment Visit and during follow-up.

### Assessment of Safety

The safety and tolerability of IV infusions of CTL-002 monotherapy and CTL-002 in combination with the defined checkpoint inhibitor will be evaluated by the incidence of AEs (all AEs will be evaluated according to NCI CTCAE v5.0), SAEs, DLTs, and use of concomitant medications. Safety assessments will include: ECGs, physical examinations including neurological examination to exclude motor neuropathy, ECOG performance status, vital signs and clinical laboratory samples (hematology, clinical chemistry, coagulation, thyroid function (thyroid stimulating hormone [TSH] and free T3), cytokines, assessment of hemoglobin A1c [HbA1c], N-terminal B-type natriuretic peptide [NT proBNP], and urinalysis).

Subjects are assessed for safety at Screening, as well as during treatment until the Safety Follow-up Visit. Thereafter safety related to the study is further captured during the follow-up of 12 months (Part A)/24 months (Part B) post-treatment.

### Vital Signs

Vital signs including systolic and diastolic BP (sitting), pulse rate, temperature, respiratory rate, and oxygen saturation should be evaluated. Additional vital sign measurements may be performed if clinically warranted.

### Physical and Neurologic Examination

A physical examination will be performed at Screening and will include examination of head, eyes, ears, nose, throat, neck, cardiovascular, chest/lungs, abdomen (including liver and spleen size), extremities, skin, and lymph nodes, as well as a brief neurologic examination to assess motor neuropathy.

### Performance Status

Performance status will be assessed at Screening according to ECOG criteria as follows:
0=Fully active, able to carry out all pre-disease activities without restrictions
1=Restricted in physically strenuous activity but ambulatory and able to carry out work of a light or sedentary nature (e.g., light housework, office work)
2=Ambulatory and capable of self-care, but unable to carry out any work activities. Up and about more than 50% of waking hours.
3=Capable of only limited self-care, confined to bed or chair more than 50% of waking hours
4=Completely disabled, cannot carry on self-care, totally confined to bed or chair
5=Death

### Cardiac Function Monitoring

Subjects will undergo a thorough monitoring for cardiac/vascular AEs and protective measures are in place to exclude subjects at risk from trial participation.

At baseline, subjects undergo an ECG, an echocardiography (or MUGA if ECHO cannot be performed) and testing for N-terminal pro b-type Natriuretic Peptid levels (NT-proBNP, heart-failure screening). Testing for NT-proBNP will be repeated every 2 weeks for 3 months and thereafter monthly or in case of any suspicion regarding cardiac/vascular damage of any type (then combined with ECG and echocardiography, again).

A single, 12-lead ECG will be performed.

All ECG monitoring is to be performed locally at the Investigator site.

The subject should be relaxed and in a recumbent or semi-recumbent position at least 5 minutes before recording an ECG.

Additional ECG testing may be performed at the Investigator's discretion if deemed clinically warranted.

### Clinical Laboratory Assessment

Samples for laboratory testing listed as below will be collected. All tests are performed locally.

Hematology/coagulation, clinical chemistry results must be available and reviewed and deemed acceptable by the Investigator or authorized designee, prior to CTL-002/PD-1/PD-L1 administration.

Clinically significant abnormal tests must be repeated to confirm the nature and degree of the abnormality. When necessary, appropriate ancillary investigations should be initiated. If the abnormality fails to resolve or cannot be explained by events or conditions unrelated to the study medication or its administration, the Medical Monitor must be consulted.

The clinical significance of an abnormal test value, within the context of the disease under study, must be determined by the Investigator which includes significant shifts from baseline within the range of normal that the Investigator considers to be clinically important.

TABLE 6
Safety Laboratory Testing

| | |
|---|---|
| Hematology | Hemoglobin, hematocrit, RBC, WBC with differential, ANC, AMC, platelet |
| | count |
| Clinical Chemistry | CRP, creatinine, LDH, calcium, electrolytes (sodium and potassium), total |
| | bilirubin, GGT, albumin, alkaline phosphatase, AST, ALT, lipase or amylase |
| | and glucose |
| Coagulation | Includes: αPTT, PT/INR |
| | INR/PT should be measured daily for any subject experiencing ALT or |
| | AST elevations ≥3 × ULN with concomitant elevation in bilirubin ≥ 2 × ULN |
| | until resolution to baseline of the liver function test abnormality. |
| | AT III |
| | D-Dimer |
| Serology | Analysis for HIV1 and HIV2, HBV, HCV, TBC, SARS-CoV-2 |
| Urinalysis | pH, ketones, specific gravity, bilirubin, protein, blood and glucose will be |
| | assessed by dipstick. |

αPTT = activated partial prothrombin time;
AMC = absolute monocyte count;
ALT = alanine aminotransferase;
ANC = absolute neutrophil count;
AST = aspartate aminotransferase;
AT III = Antithrombin III;
CRP = C-reactive protein;
GGT = gamma-glutamyltransferase;
HBV = hepatitis B virus;
HCV = hepatitis C virus;
HIV1 = human immunodeficiency virus 1;
HIV2 = human immunodeficiency virus 2;
INR = international normalized ratio;
LDH = lactate dehydrogenase;
PT = prothrombin time;
RBC = red blood cell count;
TB = tuberculosis;
ULN = upper limit of normal;
WBC = white blood cell count

### Pharmacokinetics

The PK of CTL-002 given as monotherapy and/or in combination with the defined checkpoint inhibitor will be measured from blood samples collected at the start of treatment and at various subsequent time points (Part A). Additional PK data may be evaluated in the expansion groups (Part B).

Blood samples will be taken at the start of treatment and at various subsequent time points to determine, if antibodies directed against CTL-002 may have developed.

### Monitoring of Systemic Cytokines/Chemokines (Pharmacodynamics)

Serum samples will be collected for measurement of cytokines, chemokines and other circulating biomarkers to assess pharmacodynamic effects as well as safety. Cytokines and Chemokines to be analyzed may include but are not limited to: tumor necrosis factor alpha (TNF-α), interferon (IFN)-γ, interleukin (IL)-1 β, IL-2, IL-4, IL-6, IL-8, IL-10, IL-12p70, IL-13, CXCL9 (monokine induced by gamma [MIG]) and CXCL10 (IP-10).

### Exploratory Assessments

Serum biomarker testing on specimens specifically collected for future biomedical research during this clinical trial (retention aliquots) might be conducted to identify serum factors (e.g., but not limited to metabolites, soluble growth factors, cytokines, chemokines,) important for anti-GDF-15 (CTL-002) therapy. Retrospective biomarker studies will be conducted with appropriate biostatistical design and analysis and compared to PK/Pharmacodynamic results, previously assessed biomarkers or clinical outcomes.

### Efficacy Assessment: Imaging Assessments (Local Testing)

Tumor response is evaluated according to institutional standards using RECIST V1.1 as well as imRECIST criteria. For the purposes of this study, subjects will undergo evaluation at Screening for a baseline scan and should be re-evaluated every 8 weeks beginning at Cycle 3 and/or from the End of Treatment Visit, then after this time response assessments may be performed as per local institutional guidelines until the end of the Efficacy and Survival Follow-up.

All lesions identified at Screening/baseline will be consistently followed using the unique lesion number assigned at Screening/baseline and need to be documented in the subject file and in the eCRF.

The same method of assessment (imaging modality, e.g., MRI, CT) must be used to characterize each identified and reported lesion at baseline and during all follow-up examinations for an individual subject. If there is a change in modality, then the trial site may be asked to explain the reason for the change in the eCRF. A change in modality may be considered a protocol deviation.

Each efficacy time point/visit may be completed up to a window of ±7 days.

A central reading of the images by a reading center will be performed post-hoc in addition to local reading by the investigator during the trial.

Definition of Progressive Disease According to RECIST V1.1 and iRECIST:

RECIST assessments will be used to identify subjects with possible progression of disease (Eisenhaur et al, 2009). As defined by modified RECIST V1.1 criteria for immune-based therapeutics or iRECIST criteria (Seymour et al., 2017), the date of initial potential progression by RECIST scanning will be defined as the immune unconfirmed progressive disease (iUPD) date. Subjects with an iUPD date who are stable will continue to participate in the study as planned and be reassessed for progression 4 to 8 weeks after the initial assessment. If the confirmatory assessment supports PD, the date of disease progression will be the iUPD date. If the confirmatory assessment does not support PD, the subject does not have disease progression and the iUPD date is ignored; such subjects will remain in the study as planned and continue the next imaging evaluation as planned per protocol.

Anti-tumor activity will be assessed per Investigator assessment using RECIST V1.1 and the Immune Response Criteria according to iRECIST as described below.
Contrast CT scans of the chest, abdomen and pelvis, MRI or positron emission tomography-computed tomography (PET-CT).
Disease response and disease progression will be evaluated in this study using RECIST and iRECIST criteria.
Subjects with brain and/or leptomeningeal metastases that are symptomatic or untreated or that require current therapy will not be eligible for the study. Brain imaging must not be older than 12 weeks. Results with abnormal/unexpected findings of brain MRI should be discussed with the Medical Monitor as part of the screening process.
The same method of assessment and the same technique should be used to characterize each identified and reported lesion at baseline and during follow-up. If there is a change in modality, then the trial site may be asked to explain the reason for the change in the eCRF. A change in modality may be considered a protocol deviation.

### Results

The study was initiated in December 2020 and enrolled the first patient on Dec. 9, 2020. Cohorts 1-5 have been completed without dose-limiting toxicity (DLT) and Phase 1 (Part A) of the study is completed. Note: Interim data.

### Patients and CTL-002 Treatment:

This interim report includes demographics and preliminary safety data of all 25 patients treated Phase 1 (Part A) of the CTL-002-001 trial.

TABLE 7
Patient demographics per dose cohort

| | Dose Level 1 | | Dose Level 2 | | Dose Level 3 | | Dose Level 4 | | Dose Level 5 | | | |
|---|---|---|---|---|---|---|---|---|---|---|---|---|
| | (0.3 mg/kg) | | (1.0 mg/kg) | | (3 mg/kg) | | (10.0 mg/kg) | | (20.0 mg/kg) | | Totalex | |
| | N = 3 | | N = 4* | | N = 6 | | N = 6 | | N = 6 | | N = 25 | |
| Age, years, | 72 | (66-77) | 56 | (54-61) | 59 | (54-65) | 66 | (34-79) | 62 | (56-67) | 61 | (34-79) |
| median (range) | | | | | | | | | | | | |
| Gender | | | | | | | | | | | | |
| male, | 2 | (67) | 1 | (25) | 1 | (17) | 6 | (100) | 3 | (50) | 13 | (52) |
| number (%) | | | | | | | | | | | | |
| female, | 1 | (33) | 3 | (75) | 5 | (83) | 0 | (0) | 3 | (50) | 12 | (48) |
| number (%) | | | | | | | | | | | | |
| ECOG | | | | | | | | | | | | |
| Baseline | | | | | | | | | | | | |
| 0 | 1 | (33) | 0 | (0) | 4 | (67) | 3 | (50) | 2 | (33) | 10 | (40) |
| 1 | 2 | (67) | 4 | (100) | 2 | (33) | 3 | (50) | 4 | (67) | 15 | (60) |
| Tumor type | Pleural | | Oropharyngeal | | Melanoma | | Renal CA | | Melanoma | | | |
| | Mesothelioma | | CA | | | | | | | | | |
| | Melanoma | | Cervical CA | | Melanoma | | Melanoma | | Melanoma | | | |
| | Colorectal CA | | TN-Breast CA | | Ovarian CA | | Hepatocellular CA | | Uveal | | | |
| | | | | | | | | | Melanoma | | | |
| | | | Melanoma | | Cancer of | | NSCLC | | NSCLC | | | |
| | | | | | unknown | | | | | | | |
| | | | | | primary | | | | | | | |
| | | | | | NSCLC | | Melanoma | | Endometrial | | | |
| | | | | | | | | | Ca | | | |
| | | | | | Ocular | | Cholangiocellular | | Mesothelioma | | | |
| | | | | | melanoma | | CA | | | | | |
| Prior lines of | 11 | (3.7) | 15 | (3.8) | 28 | (4.7) | 23 | (3.8) | 30 | (5.0) | 107 | (4.3) |
| therapy Number | | | | | | | | | | | | |
| (average %) | | | | | | | | | | | | |
| 1-3 | 2 | (67) | 2 | (50) | 3 | (50) | 4 | (67) | 1 | (17) | 12 | (48) |
| 4-5 | 0 | (0) | 2 | (50) | 1 | (17) | 1 | (16.5) | 3 | (50) | 7 | (28) |
| ≥6 | 1 | (33) | 0 | (0) | 2 | (33) | 1 | (16.5) | 2 | (33) | 6 | (24) |

*1 patient replaced due to delayed start of combination treatment

### Preliminary Safety and Tolerability of CTL-002:

So far monotherapy as well as the combination with nivolumab showed excellent tolerability. No dose-limiting toxicity (DLT) and no Grade 4 or 5 TEAE has occurred and no safety event of concern has been observed to date. 25 subjects have received at least 1 dose of visugromab (CTL-002) in Part A (Phase 1) of the clinical trial CTL-002-001. Adverse events were coded according to the Medical Dictionary for Regulatory activities (MedDRA).

No DLT and no≥Grade 4 AE has been reported in all subjects treated in Part A of the study.

No significant safety findings from this clinical trial occurred during the reporting period. Treatment has so far not been associated with any Adverse Events of major clinical concern and is well tolerated in all subjects treated so far in the context of treatment of advanced stage cancer. As of the time point of the interim report (cut-off date 10 Feb. 2022), 12 SAEs have been reported in 8 subjects (32.0%) in study CTL-002-001 to date. A total of 4 of these SAEs were assessed by the Investigators and/or the Sponsor as possibly related to CTL-002.

No Grade 4 (life-threatening) and no Grade 5 (fatal) AEs with relationship to CTL-002 were reported during the reporting period.

Grade 3 AEs reported in more than one patient were GGT increased and anemia (each, 2 subjects; with transfusions not permitted during DLT period). No subjects discontinued the study due to an AE prematurely.

### Biomarker Strategy and Analyses

This clinical trial explores serum- and tissue-based biomarkers. Apart from classic immune-system activation markers such as serum cytokines, specific analyses are conducted to evaluate the immunomodulatory effect of GDF-15 in the tumor microenvironment. Among other parameters, baseline GDF-15 levels, intratumoral GDF-15 levels as well as numbers and profiles of tumor-infiltrating leukocytes are analyzed prior to and under GDF-15 neutralization by CTL-002. Substantial tumor-selective GDF-15 expression was confirmed for most tumors analyzed. Tumor and tumor-stroma selective influx of mainly CD8⁺ and CD4⁺ T cells under CTL-002 dosing was observed in the majority of patients, with effect being seen from dose level 1 onwards. Preliminary analysis indicates that in several patients T cell proliferation is increased in the tumor as demonstrated by CD3/ki-67+ staining. Several tumors that at baseline are “cold tumors” characterized by rather low CD8 and CD4 counts are turned into hot tumors by increasing CD8 and CD4 counts.

Furthermore, some tumors showed reactive PD-L1 upregulation, which is an indirect sign of IFN-gamma release and an active immune response against tumor cells.

TABLE 8
List of analytes in blood, tumor and urine for PD/PK modelling,
immunological assessment and biomarker identification.

| Methods/Materials | Blood | Serum | Tumor | Urine |
|---|---|---|---|---|
| GDF-15 protein (ELISA, ECL) | − | + | − | + |
| PK total CTL-002 (ECL) | − | + | − | − |
| ADA (ECL) | − | + | − | − |
| Cytokines/Chemokines (ELISA, ECL) | − | + | − | − |
| IFN-g/Il-1b/IL-2/IL-4/IL-6/IL-8/IL-10/IL-12p70/IL13 | | | | |
| CXCL9/CXCL10 | | | | |
| Tumor infiltrating immune cells/contexture (IHC) | − | − | + | − |
| CD3/CD4/CD8/Foxp3/Ki67/ | | | | |
| GranzB/CKSox10 | | | | |
| PD-L1 | | | | |
| GDF-15 (IHC to proform) | | | | |
| Other markers optional | | | | |
| Tumor GDF-15 mRNA (FISH) (optional) | − | − | + | − |
| Molecular biomarkers | (+) | − | + | − |
| Nanostring nCounter PanCancer IO 360 ™ | | | | |
| Panel | | | | |
| Mutational load (optional) | | | | |
| Other molecular biomarkers optional (MSI, | | | | |
| mutations) | | | | |
| Sample biobanking for optional analyses (on | + | + | + | + |
| request) | | | | |

### Preliminary Response Assessment

For 6 subjects in this heavily pre-treated last-line tumor population apparent and lasting clinical benefit was observed. Three subjects experienced a confirmed partial response (PR), two currently lasting beyond 10 months duration (1× cancer of unknown primary on dose level [DL]3 and 1×mesothelioma on DL 5), the third had hepatocellular cancer with lasting, prolonged tumor regression. Three additional subjects experienced prolonged disease stabilization, 2 of them beyond 6 months (1×NSCLC and 2×melanoma). The third subject had 5.5 months of disease stabilization, then had local progression in one liver lesion compressing a vessel. Upon investigator decision study treatment was continued and local irradiation to the site added (off-study). Soon thereafter, the subject went on to a deep, confirmed partial response with abscopal effects seen in non-irradiated lesions (under continued study treatment with CTL-002 and nivolumab with permission of the sponsor). Two other subjects had minor tumor regression in isolated lesions.

In summary, in the dose escalation (Part A), 6 subjects have been treated for up to 11 months in the optional extended treatment phase and have shown lasting tumor control, including lasting, confirmed partial responses.

### CONCLUSIONS

Recent preclinical data by us and others indicate that GDF-15 potently (1) prevents T cell infiltration into the tumor microenvironment (TME) and that it (2) suppresses a potent immune response within the (TME) by other mechanisms, too. GDF-15 thus plays a key role in suppressing effective anti-tumor immune responses.

The GDFATHER (GDF-15 Antibody-mediated Effector cell Relocation) phase 2 trial explores the safety, PK and PD and preliminary antitumoral activity of the GDF-15 neutralizing antibody CTL-002 in monotherapy and combination with a checkpoint-inhibitor (CPI) in CPI-relapsed/-refractory patient populations. Anti-cachexia effects are investigated, too.

Dose level 1-5 have been completed safely with excellent tolerability and no DLT and the phase 2 part has started.

The pharmacodynamic analyses from sequential tumor biopsies (dose level 1-5) indicate a CTL-002-mediated selective T cell shift into the tumor microenvironment in the majority of samples investigated.

Preferred doses and dosage regimens for the antibodies of the invention including CTL-002 are 3, 10 or 20 mg/kg/Q2wk; a more preferred dose and dosage regimen is 10 mg/kg/Q2wk.

As reflected by the above-described favorable clinical effects which were observed in patients so far, it is expected that these dose levels are highly effective. Additionally, the dose and dosage regimen selection is based upon extensive investigations carried out by the inventors, including pharmaco-modelling and obtained PK/Pharmacodynamic data from Part A indicating complete GDF-15 neutralization at this dose in patients with all ranges of baseline GDF-15 serum levels. A review of all treatment doses and their adverse events, the PK/Pharmacodynamics observed so far, and a thorough pharmaco-modelling exercise conducted indicates full GDF-15 suppression within the tumor vasculature at 10 mg/kg of CTL-002 even at elevated baseline serum concentrations of GDF-15 of up to 10 ng/ml GDF-15 in serum, corresponding to approximately 160 ng/ml GDF-15 in immediate tumor proximity. No safety events of concern have been observed so far at this dose and no DLT occurred at this dose and there is still a >10-fold safety margin compared with the NOAEL in Non-human primates (NHP). Thus, it is expected that the above-indicated preferred doses are particularly effective and safe.

The above-indicated observations with regard to efficacy, safety, and PK/Pharmacodynamic data also show that it will be possible to advantageously administer the anti-GDF-15 antibody at a preferred dose of between 10 and 20 mg/kg, more preferably 20 mg/kg, and at a dosage regimen of at least one administration cycle, wherein the cycle is a period of three weeks with a dose of 10 mg/kg and of four weeks with a dose of 20 mg/kg and wherein said dose is to be administered at least once (i.e. preferably once) in each of the at least one cycle.

This dosage regimen has longer administration cycles of three or four weeks, but the preferred dose of between 10 and 20 mg/kg, more preferably 20 mg/kg, will allow to obtain an advantageous safety and efficacy profile similar to the preferred 10 mg/kg/Q2wk regimen. This dosage regimen is compatible with the observed PK/Pharmacodynamic profile, and a population PK/PD modeling approach used to model total CTL-002 and total GDF-15 in serum in combination with a previously developed tumor model of GDF-15 to predict systemic and tumor levels of free GDF-15 in serum for 3, 10, 20 mg/kg dosing q2w, q3w and q4w. The dosing predicted to maintain free GDF-15 in the tumor at or below the nominal healthy value of 0.5 ng/mL depends on the baseline GDF-15 with higher baseline levels require higher doses. These data support a dosing regimen of at least of 10 mg/kg every two or three weeks or at least of 20 mg/kg every 4 weeks to maintain free GDF-15 in tumor vasculature below the nominal healthy value of. 0.5 ng/mL (FIG. 5).

The above findings indicate that a treatment with anti-GDF-15 antibodies according to the invention can provide a considerable clinical benefit for cancer patients at a very low associated risk profile. Importantly, this benefit is observed in patients who had previously been refractory to some of the most advanced treatment options such as therapy with antagonists of the PD-1/PD-L1 axis (e.g., nivolumab).

### (2) Example 1

In view of the advantageous findings made in the aforementioned Reference Examples, the inventors sought to identify and further analyze cancers which are particularly responsive to a treatment with anti-GDF-15 antibodies together with aPD-1/-L1 antibodies. Based on these further analyses of the inventors, the following cancer subgroups are expected to be particularly responsive to the treatment:

For colorectal cancer (CRC), 13 selected immune-related analyses were performed in silico using mRNA expression data of numerous colorectal cancer patients. All calculations and correlation analyses were performed using the statistical software environment R. In these analyses, GDF-15 mRNA expression was correlated with 13 immunosignatures including the mRNA expression of genes relating to CD8+ cell infiltration, T-cell dysfunction, T-cell inflamed tumors, interferon gamma, inflammation, immuno-predictive scores, cytolytic activity, cytotoxic T-lymphocytes, T-cell exclusion, and the mRNA expression of the genes encoding PD-1, PD-L1 and the marker genes of CD8 cells and cytotoxic T-lymphocytes, respectively.

Remarkably, in all of these 13 immune-related analyses a significant inverse relation to GDF-15 mRNA expression was observed in the CRC patients. Additionally, a clear decrease in estimated infiltrated CD8+ T-cells could be observed from lower to higher GDF15 expression.

Similarly, for BLCA, BRCA, PAAD, LUAD, KIRC, CESC, and TGCT, in at least 10 of 13 selected immune related analyses an inverse relation to GDF-15 mRNA expression could be observed.

These findings are consistent with a strong T-cell exclusion caused by GDF-15 in the CRC, BLCA, BRCA, PAAD, LUAD, KIRC, CESC, and TGCT patients.

Therefore, therapy with the anti-GDF-15 antibodies according to the invention is expected to be particularly effective at least in CRC, BLCA, BRCA, PAAD, LUAD, KIRC, CESC, and TGCT patients due to a restoration of the infiltration of the tumors by the T-cells.

### (3) Example 2

The inventors sought to identify and further analyze cancer patients who are particularly responsive to a combination treatment comprising anti-GDF-15 antibodies and a checkpoint inhibitor across various different types of cancers. Based on these further experiments, the inventors have surprisingly found that the following patients are expected to be particularly responsive to the combination treatment:

In this respect, baseline biopsies of patients have been analysed for PD-L1 TPS and Tumor Inflammation Score (TIS) and T-cell infiltration counts.

### Pd-L1 TPS Method

For test interpretation and scoring, reference is made to the manufacturer's interpretation guideline: “VENTANA PD-L1 (SP263) Assay Staining of Non-Small Cell Lung Cancer Interpretation Guide”, version 1015317EN.

Briefly, percentage of PD-L1 positive tumor cells (TPS) was determined using an anti-PD-L1 (SP263) monoclonal antibody (Ventana, cat. #790-4905) in an immunohistochemistry (IHC) assay procedure based on formalin fixed and paraffin embedded (FFPE) sections. Tissue sections were deparaffinized and heat pretreated prior to antibody incubation to uncover all target antigens. Next, anti-PD-L1 antibody binding to PD-L1 positive cancer cells was visualized using a secondary HRP enzyme-conjugated antibody (Ventana optiView Universal DAB Detection Kit, cat. #760-700). The specific antibody-enzyme complex was visualized based on a precipitating enzyme reaction product. For determining the PD-L1 TPS, the CE-IVD cleared predictive assay (741-4905) was used which utilizes a defined cut-off of >1% membranous stained tumor cells to distinguish between negative and positive cancer tissue (e.g., NSCLC tissue) specimens. Positive cases were further subdivided by application of an additional >5% and >10% cut off, as an increased overall survival rate after treatment with Nivolumab (OPDIVO) has been observed in patients with higher PD-L1 expression levels. Slide interpretation and cut-off decisions are described in more detail in the official scoring guideline “VENTANA PD-L1 (SP263) Assay für Non-Small Cell Lung Cancer Interpretationsanleitung 2016”, version 1015317EN

For scoring, the percentage of PD-L1 positive tumor cells was determined for each staining intensity (0, 1, 2, 3) and the total percentage of stained tumor cells was calculated by summing up the percentage at the intensity 1, 2 and 3.

Analysis of clinical data revealed PD-L1 TPS as positive enrichment factor for clinical response (see FIG. 6, 7 A/B, 8).

### Tumor Inflammation Score (TIS) Method:

The TIS includes 18 functional genes (PSMB10, HLA-DQA1, HLA-DRB1, CMKLR1, HLA-E, NKG7, CD8A, CCL5, CXCL9, CD27, CXCR6, IDO1, STAT1, TIGIT, LAG3, CD274, PDCD1LG2 and CD276) known to be associated with response to PD-1/PD-L1 inhibitors pathway blockade as well as IFN-γ-responsive genes related to antigen presentation, chemokine expression, cytotoxic activity, and adaptive immune resistance genes (see Danaher, Patrick, et al, Ayers M et al.). The TIS method may be performed as briefly described in the following. First, RNA extracted from formalin fixed paraffin embedded tumor blocks was hybridized to the NanoString® PanCancer IO360™ CodeSet using nCounter® technology. Next, raw data for each sample and gene were normalized to internal ERCC controls to eliminate technical variability of the assay, and then counts were normalized to the geometric mean of endogenous housekeeping genes followed by log 2 transformation. Finally, gene expression signatures, including the TIS, were calculated as a weighted linear average of the constituent genes.

Based on the analysis of the baseline biopsies from patients, it is expected that PD-L1 and TIS show predictive value for a clinical response to a combination treatment comprising an anti-GDF-15 Ab (e.g. CTL-002) and nivolumab especially in patients being refractory for anti-PD-1/PD-L1 (see clinical responder 1-02-006, 1-03-004, 1-03-002 and long-term SD 2-01-003 having a PD-L1 TPS >1 and Tumor Inflammation Score (TIS) >7.5 in FIG. 6).

These findings of the inventors are surprising, because the present combination treatments can be used successfully even in patients who are refractory to a monotherapy with anti-PD1 or anti-PD-L1 antibodies or to combination therapies with anti-PD1 or anti-PD-L1 antibodies and other anticancer agents. Thus, such combination therapies are clinically different from a monotherapy with anti-PD1 or anti-PD-L1 antibodies or from combination therapies with anti-PD1 or anti-PD-L1 antibodies and other anticancer agents, and therefore the combination treatment of the present invention was expected to be associated with different biomarkers. Only based on the experimental results provided by the present inventors, it was surprisingly found that the combination treatment of the present invention achieves a particularly beneficial treatment outcome in patients having a PD-L1 positive cancer or an inflamed tumor as indicated based on the TIS, and this in last-line of treatment, post confirmed failure of prior anti-PD1/PD-L1 treatment.

### Determination of CD8+ T Cell Density (Cells/mm²) by Immunohistochemistry (IHC):

The CD8 protein is a glycoprotein predominantly expressed on the surface of CTLs, thereby serving as a general marker for this particular cell population. The function of CD8+ CTLs is to clear cancer cells or cells that are damaged or infected by viruses by recognizing antigen fragments presented on MHC class I on these cells. Activated CTLs release cytokines and cytotoxic enzymes, such as granzyme B and perforin, that leads to killing of the cancerous or infected cells. The primary (monoclonal) antibodies (Monoclonal Mouse Anti-Human CD8, clone C8/144B) applied in the herein described multiplex IHC assay procedure bound to their respective antigen in FFPE tissue sections (thickness: 4 μm). To uncover all target antigens, tissue sections were deparaffinized and heat pretreated prior to antibody incubation. Binding of the specific antibody could be visualized using a secondary horseradish peroxidase (HRP)—or alkaline phosphatase (AP)—enzyme-conjugated antibody, in most cases subsequent to application of one or more linker antibodies, hydroxyquinoxaline (HQ)—or nitropyrazole (NP)—conjugated, thus allowing for amplification of the resulting signal. The enzyme coupled to the secondary antibody catalyzed a chromogenic precipitate-forming reaction at the binding site of the actual primary antibody. Each step was incubated. After each incubation step, the VENTANA DISCOVERY ULTRA automated slide stainer washed the sections to stop the respective reaction and to remove unbound material that would have hindered the desired reaction in subsequent steps. Prior to application of the next primary antibody, the previous reagents were removed by heat incubation (“stripping”), leaving only the insoluble precipitate on the slide. For quantitative evaluation of stained slides, the Visiopharm digital image analysis software VIS 2019.02 (TID 001673) was used. Stained slides were scanned at a magnification of 20× using the Aperio ScanScope XT (Leica) prior to image analysis. The final workflow allows detection of not only the positive staining for the single biomarkers but is also able to discriminate between the tissue compartments of tumor, normal tissue and peri- and intratumoral stroma. To achieve this, an additional guidance staining with the tumor-specific markers panCK (solid tumors) or SOX10 (malignant melanoma) was introduced, allowing for generation of virtual overlay images by alignment of consecutive sections and transfer of information from one slide to the other. The final readout included area measurements (mm²) per tissue compartment, cell counts per cell type per compartment, density calculations (cell counts per mm²) and distance measurements for biologically relevant combinations of cell types.

### Determination of CD3+ T Cell Density (Cells/mm²) by IHC:

CD3+ T cell density was determined in a ULTIVUE® Multiplex Immunofluorescence Assay (UltiMapper 1/0 T-act Panel CD3, Granzyme B, Ki67 and a panCK/SOX10) in 4 μm thick FFPE tissue sections, using the UltiMapper® I/O T-act kit by ULTIVUE (p/n ULT20110) stained on Leica Bond RX, digitized as full-slide images using ZEISS Axioscan Z1 and subsequent digital analysis in Visiopharm.

Baseline biopsies from clinical responder 1-02-006, 1-03-004, 1-03-002, 1-02-036 and 1-06-005 and long-term SD 2-01-003 show PD-L1 TPS >1 and CD8+ T cell density of >300 cells/mm² (FIG. 7 A, B). It is therefore expected that not only the TPS, but also the CD8+ T cell density has predictive value for the treatment of human patients with anti-GDF-15 antibodies such as CTL-002, e.g., when administered in combination with anti-PD-1 or anti-PD-L1 antibodies such as Nivolumab to anti-PD-1 and/or anti-PD-L1 refractory patients. The same applies to the CD3+ T cell density (FIG. 8).

In patients treated with anti-GDF15, an about 2-4 fold change in CD8+ cell influx is observed on days 14 and 28 on average as compared to baseline (Bsl), indicating that anti-GDF15 antibodies increase the percentage of CD8+ cells in the tumors relative to baseline (FIG. 9, upper panel). Similarly, immune cell analysis in biopsies from baseline (bsl), day 14 and day 28 of patients revealed an increase in proliferating CD3+ki67+ T cells (FIG. 9, lower panel) under treatment.

In summary, analysis of clinical data revealed inter alia PD-L1 TPS as positive enrichment factor for clinical response (see FIG. 6, 7A/B, 8).

Moreover, clinical data also revealed that patient selection by PD-L1 TPS alone or a combination of PD-L1 TPS with any of CD8 cell density, CD3 cell density or TIS enriched for clinical responder.

For example, out of 14 bladder cancer patients, 2 patients show PD-L1 TPS >5 and both patients show PR. Similarly, out of 14 bladder cancer patients, 7 patients show PD-L1 TPS >1 and 28.6% (2 out of 7) thereof show PR.

Moreover, out of 14 bladder cancer patients, 2 patients show PD-L1 TPS >5 and CD8 cell density >300 cells/mm² and both patients show PR. Similarly, out of 14 bladder cancer patients, 3 patients show PD-L1 TPS >1 and CD8 cell density >300 cells/mm² and 66.7% (2 out of 3) thereof show PR.

### Sequences

SEQ ID No: 1(Heavy Chain CDR1 Region Peptide Sequence of monoclonal anti-human GDF-15 antibody):

GFSLSTSGMG

SEQ ID No: 2 (Heavy Chain CDR2 Region Peptide Sequence of monoclonal anti-human GDF-15 antibody):

IYWDDDK

SEQ ID No: 3 (Heavy Chain CDR3 Region Peptide Sequence of monoclonal anti-human GDF-15 antibody):

ARSSYGAMDY

SEQ ID No: 4 (Light Chain CDR1 Region Peptide Sequence of monoclonal anti-human GDF-15 antibody):

QNVGTN

Light Chain CDR2 Region Peptide Sequence of monoclonal anti-human GDF-15 antibody:

SAS

SEQ ID No: 5 (Light Chain CDR3 Region Peptide Sequence of monoclonal anti-human GDF-15 antibody):

QQYNNFPYT

SEQ ID No: 6 (heavy chain variable domain of monoclonal anti-human GDF-15 antibody):

QITLKESGPTLVKPTQTLTLTCTFSGFSLSTSGMGVSWIRQPPGKGLEW
LAHIYWDDDKRYNPTLKSRLTITKDPSKNQVVLTMTNMDPVDTATYYCA
RSSYGAMDYWGQGTLVTVSSASTKGP

SEQ ID No: 7 (light chain variable domain of monoclonal anti-human GDF-15 antibody):

DIVLTQSPSFLSASVGDRVTITCKASQNVGTNVAWFQQKPGKSPKALIY
SASYRYSGVPDRFTGSGSGTEFTLTISSLQPEDFAAYFCQQYNNFPYTF
GGGTKLEIKRT

SEQ ID No: 8 (heavy chain of monoclonal anti-human GDF-15 antibody CTL-002 without the leader peptide sequence):

QITLKESGPTLVKPTQTLTLTCTFSGFSLSTSGMGVSWIRQPPGKGLEW
LAHIYWDDDKRYNPTLKSRLTITKDPSKNQVVLTMTNMDPVDTATYYCA
RSSYGAMDYWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLG

SEQ ID No: 9 (light chain of monoclonal anti-human GDF-15 antibody CTL-002 without the leader peptide sequence):

DIVLTQSPSFLSASVGDRVTITCKASQNVGTNVAWFQQKPGKSPKALIY
SASYRYSGVPDRFTGSGSGTEFTLTISSLQPEDFAAYFCQQYNNFPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

SEQ ID NO: 10 (heavy chain variable domain of anti-human GDF-15 antibody H1L5):

QITLKESGPTLVKPTQTLTLTCTFSGFSLSTSGMGVSWIRQPPGKGLEW
LAHIYWDDDKRYNPTLKSRLTITKDPSKNQVVLTMTNMDPVDTATYYCA
RSSYGAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG

SEQ ID NO: 11 (light chain variable domain of anti-human GDF-15 antibody H1L5):

DIVLTQSPSFLSASVGDRVTITCKASQNVGTNVAWFQQKPGKSPKALIY
SASYRYSGVPDRFTGSGSGTEFTLTISSLQPEDFAAYFCQQYNNFPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Discontinuous epitope (EVQVTMCIGACPSQFR (SEQ ID NO: 12) ---38 amino acids ---

(SEQ ID NO: 13))
TDTGVSLQTYDDLLAKDCHCI

Sequences shown in FIG. 3:
GDF-15 human: SEQ ID NO: 14
GDF-15 macfa: SEQ ID NO: 15
GDF-15 mouse: SEQ ID NO: 16
GDF-15 rat: SEQ ID NO: 17
Partial consensus CTL-002 epitope (b): SEQ ID NO: 18

SEQ ID NO: 19 (light chain variable region domain of anti-mGDF-15 antibody)

DIVMTQSPSSLSVSAGEKVTMSCRSSQSLLWKHGYNYLDWYQQKPGQPP
KLLIYLDRNRAHGVPDRFTGSGSGTDFTLTISSVQAEDLAVYYCMQSFE
TPITFGGGTKLEIKRADAAPTVSIFPPSSEQLTSGGASVVCFLNNFYPK
DINVKWKIDGSERQNGVLNSWTDQDSKDSTYSMSSTLTLTKDEYERHNS
YTCEATHKTSTSPIVKSFNRNEC

SEQ ID NO: 20 (heavy chain variable region domain of anti-mGDF-15 antibody)

QVQLQQPGAELVKPGASVKMSCKASGYPFEGWYIHWVKQRPGQGLEWMG
WNNPRTGLTNHAQKFQGKVTMTRDTSSSTAYMQLSSLTSEDSAVYYCAR
GVGADAAFDIWGQGTTLTVSSAKTTPPSVYPLAPGSAAQTNSMVTLGCL
VKGYFPEPVTVTWNSGSLSSGVHTFPAVLQSDLYTLSSSVTVPSSTWPS
ETVTCNVAHPASSTKVDKKIVPRDCGCKPCICTVPEVSSVFIFPPKPKD
VLTITLTPKVTCVVVAISKDDPEVQFSWFVDDVEVHTAQTQPREEQFNS
TFRSVSELPIMHQDWLNGKEFKCRVNSAAFPAPIEKTISKTKGRPKAPQ
VYTIPPPKEQMAKDKVSLTCMITDFFPEDITVEWQWNGQPAENYKNTQP
IMDTDGSYFIYSKLNVQKSNWEAGNTFTCSVLHEGLHNHHTEKSLSHSP
GK

### REFERENCES

Abulizi P, Loganathan N, Zhao D, et al. Growth Differentiation Factor-15 Deficiency Augments Inflammatory Response and Exacerbates Septic Heart and Renal Injury Induced by Lipopolysaccharide Sci Rep. 2017; 7(1):1-10.
Böttner, Martina, Martin Laaff, Birgit Schechinger, Gudrun Rappold, Klaus Unsicker, and Clemens Suter-Crazzolara. 1999. “Characterization of the Rat, Mouse, and Human Genes of Growth/Differentiation Factor-15/Macrophage Inhibiting Cytokine-1 (GDF-15/MIC-1).” Gene 237 (1): 105-11.
Chung H K, Kim J T, Kim H W, et al. GDF15 deficiency exacerbates chronic alcohol- and carbon tetrachloride-induced liver injury. Sci Rep. 2017; 7(1):1-13. doi:10.1038/s41598-017-17574-w
Eisenhauer E A, Therasse P, Bogaerts J, et al. New Response Evaluation Criteria in Solid Tumours: Revised RECIST Guideline (Version 1.1). Eur J Cancer. 2009 January; 45(2):228-47.
Emmerson, Paul J., Feng Wang, Yong Du, Qian Liu, Richard T. Pickard, Malgorzata D. Gonciarz, Tamer Coskun, et al. 2017. “The Metabolic Effects of GDF15 Are Mediated by the Orphan Receptor GFRAL.” Nature Medicine 23 (10): 1215-19. https://doi.org/10.1038/nm.4393.
Johnen H, Lin S, Kuffner T, et al. Tumor-induced anorexia and weight loss are mediated by the TGF-β superfamily cytokine MIC-1. Nat Med. 2007; 13(11):1333-1340. doi:10.1038/nm1677
Kempf, Tibor, Alexander Zarbock, Christian Widera, Stefan Butz, Anika Stadtmann, Jan Rossaint, Matteo Bolomini-Vittori, et al. 2011. “GDF-15 Is an Inhibitor of Leukocyte Integrin Activation Required for Survival after Myocardial Infarction in Mice.” Nature Medicine 17 (5): 581-88. https://doi.org/10.1038/nm.2354.
Selby, Mark J., John J. Engelhardt, Robert J. Johnston, Li-Sheng Lu, Minhua Han, Kent Thudium, Dapeng Yao, et al. 2016. “Preclinical Development of Ipilimumab and Nivolumab Combination Immunotherapy: Mouse Tumor Models, In Vitro Functional Studies, and Cynomolgus Macaque Toxicology.” Edited by Aamir Ahmad. PLOS ONE 11 (9): e0161779. https://doi.org/10.1371/journal.pone.0161779.
Seymour et al., iRECIST: guidelines for response criteria for use in trials testing immunotherapeutics, Lancet Oncol. 2017 March; 18(3): e143-e152. 18(3): e143-e152.
Tong S, Marjono B, Brown D A, et al. Serum concentrations of macrophage inhibitory cytokine 1 (MIC 1) as a predictor of miscarriage. Lancet. 2004; 363(9403):129-130. doi:10.1016/S0140-6736(03)15265-8
Tsai, Vicky W. W., Yasmin Husaini, Amanda Sainsbury, David A. Brown, and Samuel N. Breit. 2018. “The MIC-1/GDF15-GFRAL Pathway in Energy Homeostasis: Implications for Obesity, Cachexia, and Other Associated Diseases.” Cell Metabolism 28 (3): 353-68. https://doi.org/10.1016/j.cmet.2018.07.018.
van den Boorn, Jasper G., and Gunther Hartmann. 2013. “Turning Tumors into Vaccines: Co-Opting the Innate Immune System.” Immunity 39 (1): 27-37. https://doi.org/10.1016/j.immuni.2013.07.011.
Vaupel, Peter. 2004. “Tumor Microenvironmental Physiology and Its Implications for Radiation Oncology.” Seminars in Radiation Oncology 14 (3): 198-206. https://doi.org/10.1016/j.semradonc.2004.04.008.
Wang M, Yao L C, Cheng M, Cai D, Martinek J, Pan C X, et al. Humanized mice in studying efficacy and mechanisms of PD-1-targeted cancer immunotherapy. FASEB J. 2018; 32(3):1537-49.
Welsh, John B., Lisa M. Sapinoso, Suzanne G. Kern, David A. Brown, Tao Liu, Asne R. Bauskin, Robyn L. Ward, et al. 2003. “Large-Scale Delineation of Secreted Protein Biomarkers Overexpressed in Cancer Tissue and Serum.” Proceedings of the National Academy of Sciences 100 (6): 3410-15. https://doi.org/10.1073/PNAS.0530278100.
Wischhusen J, Melero I, and Fridman W. GDF-15: From Biomarker to Novel Targetable Immune Checkpoint. Front. Immunol. Accepted 23 Apr. 2020. doi: 10.3389/fimmu.2020.00951.
Wollert K C, Kempf T, Giannitsis E, et al. An Automated Assay for Growth Differentiation Factor 15. J Appl Lab Med An AACC Publ. 2018; 1(5):510-521. doi:10.1373/jalm.2016.022376
Jerby-Arnon et al. (2018), Cell. 175(4):984-997)
Trujillo et al. (2018), Cancer Immunol Res. 6(9):990-1000
Spranger et al. (2016), Proc Natl Acad Sci USA. 113(48):E7759-E7768)
Roelands et al., Int J Mol Sci. 2017 October; 18(10): 2229
Guinney et al. (Nat Med. 2015 Nov.; 21(11):1350-6. doi: 10.1038/nm.3967. Epub 2015 Oct. 12)
McConkey et al., Curr Oncol Rep. 2018 Aug. 20; 20(10):77
Allison et al. (J Clin Oncol. 2020 Apr. 20; 38(12):1346-1366. doi: 10.1200/JCO.19.02309. Epub 2020 Jan. 13)
Pu et al. (Breast Cancer Res Treat. 2020 January; 179(1):197-206)
Inamura (Oncotarget. 2018 Feb. 16; 9(18):14723-14737. doi: 10.18632/oncotarget.24515. eCollection 2018 Mar. 6)
Augustin et al. (Front Oncol. 2020 Sep. 15; 10:1751. doi: 10.3389/fonc.2020.01751. eCollection 2020)
Danaher, Patrick, et al. “Pan-cancer adaptive immune resistance as defined by the Tumor Inflammation Signature (TIS): results from The Cancer Genome Atlas (TCGA).” Journal for immunotherapy of cancer 6.1 (2018): 1-17.
Ayers M, Lunceford J, Nebozhyn M, Murphy E, Loboda A, Kaufman D R, Albright A, Cheng J D, Kang S P, Shankaran V, Piha-Paul S A, Yearley J, Seiwert T Y, Ribas A, McClanahan T K. IFN-γ-related mRNA profile predicts clinical response to PD-1 blockade. J Clin Invest. 2017 Aug. 1; 127(8):2930-2940. doi: 10.1172/JC191190. Epub 2017 Jun. 26. PMID: 28650338; PMCID: PMC5531419.
Tumeh et al., Nature. 2014 Nov. 27; 515(7528):568-71.
Yarchoan et al., JCI Insight. 2019 Mar. 21; 4(6):e126908.
Chen et al., Exp Hematol Oncol. 2020 Aug. 3; 9:17. doi: 10.1186/s40164-020-00173-3. eCollection 2020.
WO 2022/101263
US 2020/0055930

### INDUSTRIAL APPLICABILITY

The anti-GDF-15 antibody used in methods for the treatment of cancer in human patients can be industrially manufactured and sold as products for the itemed methods and uses, in accordance with known standards for the manufacture of pharmaceutical products. Accordingly, the present invention is industrially applicable.

1. A method of treating cancer in a human patient, the method comprising administering to the patient a combination of an anti-GDF-15 antibody and at least one checkpoint inhibitor, wherein the human patient has a PD-L1 positive cancer and/or has a cancer comprising an inflamed tumor.

2. The method according to claim 1, wherein the cancer:
(a) is PD-L1 positive as determined by an immunohistochemistry (HC) assays;
(b) is PD-L1 positive as determined by the percentage of PD-L1 positive cancer cells;
(c) is PD-L1 positive as determined by the percentage of cancer cells showing staining for the presence of PD-L1 on the cell surface of at least 1 in an immunohistochemistry (IHC) assay;
(d) is PD-L1 positive as determined by the summed-up percentage of PD-L1 positive cancer cells and PD-L1 positive cancer-infiltrating cells;
(e) is a solid cancer; and/or
(f) is selected from the group consisting of the group consisting of colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP) and breast carcinoma.

3-5. (canceled)

6. The method according to claim 1, wherein the inflamed tumor:
(a) is a T-cell inflamed tumor;
(b) is a tumor which contains CD3+ cells;
(c) contains CD3+ cells as determinable by an immunohistochemistry (IHC) assay, and wherein the IHC assay uses an anti-CD3 antibody as primary antibody;
(d) is a tumor having an average CD3+ cell density of >300 cells/mm² in immunohistochemical tissue sections having a thickness of 4 m, as assessed by the immunohistochemistry (IHC) assay;
(e) is a tumor which contains CD8+ cells;
(f) contains CD8+ cells as determinable by an immunohistochemistry (IHC) assay, and wherein the IHC assay uses an anti-CD8 antibody as primary antibody;
(g) is a tumor having an average CD8+ cell density of >300 cells/mm² in immunohistochemical tissue sections having a thickness of 4 m, as assessed by the immunohistochemistry (IHC) assay; and/or
(h) is inflamed as indicated by its tumor inflammation signature (TIS).

7.-13. (canceled)

14. The method according to claim 6, wherein the TIS:
(a) is based on the expression of at least one, preferably all, of the marker genes selected from the group consisting of PSMB10, HLA-DQA1, HLA-DRB1, CMKLR1, HLA-E, NKG7, CD8A, CCL5, CXCL9, CD27, CXCR6, IDO1, STAT1, TIGIT, LAG3, CD274, PDCD1LG2 and CD276;
(b) is a linear combination of all of the marker genes in part (a), calculated as
TIS=Σᵢ₌₁¹⁸×ᵢwᵢ,
where xᵢ is the i^{th} gene's log 2-transformed, normalized expression level and wᵢ is a predefined weight above zero; and/or
(c) is greater than 7.5.

15.-16. (canceled)

17. The method according to claim 1, wherein the human patient is an anti-PD-1 and/or anti-PD-L1 relapsed and/or refractory patient.

18. The method according to claim 1, wherein the checkpoint inhibitor is selected from the group consisting of an anti-PD-1 antibody or a PD-1-binding fragment thereof, an anti-PD-L1 antibody or a PD-L1-binding fragment thereof, an anti-CD40 antibody or a CD40-binding fragment thereof, an anti-LAG-3 antibody or a LAG-3-binding fragment thereof, an anti-TIM-3 antibody or a TIM-3-binding fragment thereof, an anti-TIGIT antibody or a TIGIT-binding fragment thereof, an anti-CTLA4 antibody or a CTLA4-binding fragment thereof, and combinations thereof.

19.-21. (canceled)

22. The method according to claim 1, wherein the anti-GDF-15 antibody comprises a heavy chain variable domain comprising a CDR1 region represented by an amino acid sequence shown in SEQ ID NO: 1, a CDR2 region represented by an amino acid sequence shown in SEQ ID NO: 2 and a CDR3 region represented by an amino acid sequence shown in SEQ ID NO: 3 and a light chain variable domain comprising a CDR1 region represented by an amino acid sequence shown in SEQ ID NO: 4, a CDR2 region represented by an amino acid sequence of ser-ala-ser and a CDR3 region represented by an amino acid sequence shown in SEQ ID NO: 5.

23. The method according to claim 22, wherein the anti-GDF-15 antibody:
(a) comprises a heavy chain variable domain comprising the amino acid sequence represented by SEQ ID NO: 6 or an amino acid sequence having at least 90% identity to the amino acid sequence shown in SEQ ID NO: 6; and a light chain variable domain comprising the amino acid sequence represented by SEQ ID NO: 7 or an amino acid sequence having at least 90% identity to the amino acid sequence shown in SEQ ID NO: 7; or
(b) comprises a heavy chain comprising the amino acid sequence represented by SEQ ID NO: 8 or an amino acid sequence having at least 90% identity to the amino acid sequence shown in SEQ ID NO: 8, and a light chain comprising the amino acid sequence represented by SEQ ID NO: 9 or an amino acid sequence having at least 90% identity to the amino acid sequence shown in SEQ ID NO: 9.

24.-25. (canceled)

26. The method according to claim 1, wherein the anti-GDF-15 antibody is administered to the human patient
at a dose of between 3 and 20 mg/kg and at a dosage regimen of at least one administration cycle, wherein the cycle is a period of two, three, or four weeks, and wherein said dose is administered at least once in each of the at least one cycle,
and wherein the cancer is selected from the group consisting of colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP) and breast carcinoma.

27.-34. (canceled)

35. The method according to claim 1, wherein the method also treats cancer-cachexia.

36. The method according to claim 26, wherein the anti-GDF-15 antibody is administered at a dose of 20 mg/kg and the administration cycle is a period of four weeks.

37. The method according to claim 26, wherein the anti-GDF-15 antibody is administered at a dose of 10 mg/kg and the administration cycle is a period of three weeks or two weeks.

38. (canceled)

39. The method according to claim 1, wherein the anti-GDF-15 antibody is produced by expression in CHO cells.

40. The method according to claim 1, wherein the checkpoint inhibitor:
(a) is administered in the same dosage regimen as the anti-GDF-15 antibody;
(b) is administered prior to the administration of anti-GDF-15 antibody;
(c) is administered within 120 min prior to the administration of anti-GDF-15 antibody; and/or
(d) is administered within 30 min prior to the administration of anti-GDF-15 antibody.

41. (canceled)

42. The method according to claim 1, wherein the dose of the anti-GDF-15 antibody is administered intravenously.

43. (canceled)

44. A method of treating a human patient diagnosed with cancer comprising the steps of:
(a) analyzing whether the cancer is a PD-L1 positive cancer and/or is a cancer comprising an inflamed tumor,
(b) predicting the clinical outcome of the patient for the treatment based on the analysis, and
(c) treating the human patient by administering a combination of an anti-GDF-15 antibody and at least one checkpoint inhibitor.

45. The method according to claim 43, wherein the clinical outcome comprises response, complete response, partial response, stable disease, progressive disease and/or survival of a human cancer patient.

46. The method according to claim 44, wherein the patient is predicted to show improved clinical outcome if the cancer is PD-L1 positive or comprises an inflamed tumor.

47.-60. (canceled)

61. The method according to claim 1, wherein the PD-L1 positive cancer has a percentage of cancer cells showing staining for the presence of PD-L1 on the cell surface of at least 5 in an immunohistochemistry (IHC) assay.

62. The method according to claim 61, wherein the cancer is bladder cancer.


SEQUENCE LISTING (21 sequences)

SEQ ID NO: 1 - protein, 10 aa, synthetic construct
  REGION 1..10: note = Heavy Chain CDR1 Region Peptide Sequence of monoclonal anti-human GDF-15 antibody
GFSLSTSGMG

SEQ ID NO: 2 - protein, 7 aa, synthetic construct
  REGION 1..7: note = Heavy Chain CDR2 Region Peptide Sequence of monoclonal anti-human GDF-15 antibody
IYWDDDK

SEQ ID NO: 3 - protein, 10 aa, synthetic construct
  REGION 1..10: note = Heavy Chain CDR3 Region Peptide Sequence of monoclonal anti-human GDF-15 antibody
ARSSYGAMDY

SEQ ID NO: 4 - protein, 6 aa, synthetic construct
  REGION 1..6: note = Light Chain CDR1 Region Peptide Sequence of monoclonal anti-human GDF-15 antibody
QNVGTN

SEQ ID NO: 5 - protein, 9 aa, synthetic construct
  REGION 1..9: note = Light Chain CDR3 Region Peptide Sequence of monoclonal anti-human GDF-15 antibody
QQYNNFPYT

SEQ ID NO: 6 - protein, 124 aa, synthetic construct
  REGION 1..124: note = heavy chain variable domain of monoclonal anti-human GDF-15 antibody
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSSASTKGP

SEQ ID NO: 7 - protein, 109 aa, synthetic construct
  REGION 1..109: note = light chain variable domain of monoclonal anti-human GDF-15 antibody
DIVLTQSPSFLSASVGDRVTITCKASQNVGTNVAWFQQKPGKSPKALIYSASYRYSGVPDRFTGSGSGTEFTLTISSLQPEDFAAYFCQQYNNFPYTFGGGTKLEIKRT

SEQ ID NO: 8 - protein, 444 aa, synthetic construct
  REGION 1..444: note = heavy chain of monoclonal anti-human GDF-15 antibody CTL-002 without the leader peptide sequence
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 214 aa, synthetic construct
  REGION 1..214: note = light chain of monoclonal anti-human GDF-15 antibody CTL-002 without the leader peptide sequence
DIVLTQSPSFLSASVGDRVTITCKASQNVGTNVAWFQQKPGKSPKALIYSASYRYSGVPDRFTGSGSGTEFTLTISSLQPEDFAAYFCQQYNNFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 447 aa, synthetic construct
  REGION 1..447: note = heavy chain variable domain of anti-human GDF-15 antibody H1L5
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, synthetic construct; identical to SEQ ID NO: 9
  REGION 1..214: note = light chain variable domain of anti-human GDF-15 antibody H1L5

SEQ ID NO: 12 - protein, 16 aa, Homo sapiens
  REGION 1..16: note = Discontinuous epitope
EVQVTMCIGACPSQFR

SEQ ID NO: 13 - protein, 21 aa, Homo sapiens
  REGION 1..21: note = Discontinuous epitope
TDTGVSLQTYDDLLAKDCHCI

SEQ ID NO: 14 - protein, 89 aa, Homo sapiens
  REGION 1..89: note = GDF-15 HUMAN
LEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 15 - protein, 89 aa, Macaca fascicularis
  REGION 1..89: note = GDF-15 MACFA
LEDLGWADWVLSPREVQVTMCIGACPSQFREANMHAQIKMNLHRLKPDTVPAPCCVPASYNPMVLIQKTDTGVSLQTYDDLLAKDCHCV

SEQ ID NO: 16 - protein, 89 aa, Mus musculus
  REGION 1..89: note = GDF-15 MOUSE
LEDLGWSDWVLSPRQLQLSMCVGECPHLYRSANTHAQIKARLHGLQPDKVPAPCCVPSSYTPVVLMHRTDSGVSLQTYDDLVARGCHCA

SEQ ID NO: 17 - protein, 89 aa, Rattus norvegicus
  REGION 1..89: note = GDF-15 RAT
LEDLGWSDWVLSPRQLQLSMCVGECPHLYRSANTHALIKARLHGLQPDRVPAPCCVPSSYTPVVLMHRTDSGVSLQTYDDLVAQGCHCA

SEQ ID NO: 18 - protein, 20 aa, unidentified
  VARIANT 3: note = X can be T or S
  VARIANT 14: note = X can be V or L
  VARIANT 17: note = X can be G or D
  REGION 1..20: note = Partial consensus CTL-002 epitope (b)
TDXGVSLQTYDDLXAKXCHC

SEQ ID NO: 19 - protein, 219 aa, synthetic construct
  REGION 1..219: note = light chain variable region domain of anti-mGDF-15 antibody
DIVMTQSPSSLSVSAGEKVTMSCRSSQSLLWKHGYNYLDWYQQKPGQPPKLLIYLDRNRAHGVPDRFTGSGSGTDFTLTISSVQAEDLAVYYCMQSFETPITFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 443 aa, synthetic construct
  REGION 1..443: note = heavy chain variable region domain of anti-mGDF-15 antibody
QVQLQQPGAELVKPGASVKMSCKASGYPFEGWYIHWVKQRPGQGLEWMGWNNPRTGLTNHAQKFQGKVTMTRDTSSSTAYMQLSSLTSEDSAVYYCARGVGADAAFDIWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 21 - protein, 16 aa, unidentified
  VARIANT 1: note = X can be Q or E
  VARIANT 2: note = X can be L or V
  VARIANT 4: note = X can be L or V
  VARIANT 5: note = X can be S or T
  VARIANT 8: note = X can be V or I
  VARIANT 10: note = X can be E or A
  VARIANT 13: note = X can be H or S
  VARIANT 14: note = X can be L or Q
  VARIANT 15: note = X can be Y or F
  REGION 1..16: note = Partial Consensus CTL-002 epitope (a)
XXQXXMCXGXCPXXXR